(19) United States
(12) Patent Application Publication
(10) Pub. No.: US 2025/0162994 A1
(43) Pub. Date: May 22, 2025

(54) DUAL LSD1/HDAC INHIBITORS

(71) Applicant: JUBILANT EPICORE LLC, Bedminster, NJ (US)

(72) Inventors: Naveen SADHU, Karnataka, Bangalore (IN); Dhanalakshmi SIVANANDHAN, Karnataka, Bangalore (IN); Luca RASTELLI, Bedminster, NJ (US); Chandru GAJENDRAN, Karnataka, Bangalore (IN); Rajagopal SRIDHARAN, Karnataka, Bangalore (IN)

(21) Appl. No.: 18/842,771

(22) PCT Filed: Mar. 10, 2023
(86) PCT No.: PCT/IN2023/050233

§ 371 Date: Aug. 29, 2024

Related U.S. Application Data
(60) Provisional application No. 63/368,342, filed on Jul. 13, 2022.

(30) Foreign Application Priority Data
Mar. 10, 2022 (IN) 202241013165
Oct. 4, 2022 (IN) 202241057045

(51) Int. Cl. C07D 231/12; A61K 31/397; A61K 31/415; A61K 31/4192; A61K 31/445; A61K 31/454; A61K 31/4545; A61K 31/506; A61K 45/06; A61P 35/00; C07D 205/04; C07D 211/34; C07D 249/04; C07D 401/12; C07D 413/12

(52) U.S. Cl. CPC C07D 231/12; A61K 31/397; A61K 31/415; A61K 31/4192; A61K 31/445; A61K 31/454; A61K 31/4545; A61K 31/506; A61K 45/06; A61P 35/00; C07D 205/04; C07D 211/34; C07D 249/04; C07D 401/12; C07D 413/12

(57) ABSTRACT

The present disclosure provides a compound of Formula (I), its analogs, tautomeric forms, stereoisomers, enantiomers, diastereomers and pharmaceutically acceptable salts thereof. The present disclosure also provides a process for preparing the compounds of Formula (I), its analogs, tautomeric forms, stereoisomers, enantiomers, diastereomers and pharmaceutically acceptable salts thereof. The compounds of Formula (I) modulates MYC or coREST, in a subject in need thereof. The compounds of Formula (I) inhibits HDAC enzymes or LSD1 enzymes, or both HDAC enzymes and LSD1 enzymes. The compound of Formula (I) is an epigenetic modulating agent with a novel mechanism of action that target MYC amplification in multiple neuroendocrine tumors types.

### FIELD OF THE DISCLOSURE

The present disclosure relates in general, to the field of pharmaceutical compounds, more particularly to the compounds of Formula (I) as inhibitors of lysine specific demethylase (LSD1) or histone deacetylase (HDAC) or both LSD1 and HDAC, specifically, the enantiomeric form and its related compounds. In particular, substantially pure enantiomeric form of the compound of Formula (I), as a potent LSD1/HDAC inhibitors for the treatment of a disease and/or prevention of a condition associated with it. The present disclosure further relates to LSD1/HDAC dual inhibitor of compound of Formula (I), an epigenetic modulating agent with a novel mechanism of action that target MYC amplification in multiple neuroendocrine tumors types.

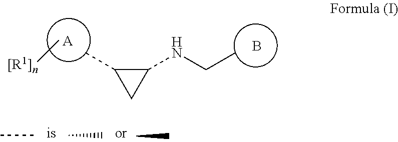

### BACKGROUND

Lysine specific demethylase 1 (LSD1) and Histone deacetylase (HDAC) are epigenetic proteins which are found to be associated with several diseases, including cancer. LSD1 is actively involved in catalysing the demethylation of histone 3 to control gene expression. LSD1 take part in various cellular processes and is involved in embryonic development, cell proliferation, and metastasis. Further, the HDAC enzyme family plays a crucial role in the modulation of several biological processes, including transcription and cell cycle. Several structural classes of HDAC inhibitors have been identified and are reviewed in Marks et al., J. Natl. Cancer Inst., 2000, 92, 1210-1215; L. Zhang, et. al., Medicinal Research Reviews, 2015, 35, 63-84; P. K. Agrawala, et. al., HOAJ Biology 2013, 2, 1-8. Other compounds that can inhibit HDAC activity are Trichostatin A (TSA), PXD101, Tropoxin (TPX), Sodium butyrate (NaB), Sodium valproate (VPA), Cyclic hydroxamic acid containing peptides (CHAPs), Depsipeptide FK-228, MGCDO103 and MS-275. The above mentioned inhibitors can also de-repress tumor suppressor genes (e.g. p21wafl/cf1), resulting in antiproliferative effects in vitro and anti-tumor effects in vivo. At present, four HDAC inhibitors have been approved by FDA for the treatment of various cancers. Vorinostat, Istodax and Belinostat have been approved for the treatment of Cutaneous T-Cell Lymphoma and Panobinostat has been approved for the treatment of multiple myeloma.

Lysine methyl transferases and lysine demethylases are involved in the modulation of histone methylation. Lysine demethylases (LSD1 and LSD2) are known to remove methyl group from mono and dimethylated Lys4 of histone H3 (H3K4me1/2) through flavin adenine dinucleotide (FAD) dependent enzymatic oxidation and releasing formaldehyde as the byproduct. LSD1 mediated demethylation is not restricted to histones; other non-histone substrates such as p53, STAT3, E2F1, and MYPT1 are also demethylated leading to a change in cellular functions.

LSD1 is overexpressed in various cancer cells and tissues, neuroblastoma, prostate cancer, breast cancer, leukemia, lung cancer and bladder cancer cells. It is known that either inhibition of LSD1 with small molecule or by RNAi is associated with inhibition of cancer cell growth by modulating pro-survival gene expression and p53 transcriptional activity. J. W. Hφjfeldt, et. al., Nature Drug Discovery, 12, 917-930, 2013, Manfred Jung and et. al., Clinical Epigenetics (2016) 8:57 identifies novel inhibitors for LSD1/HDAC.

Another recent report suggests that a cross talk between LSD1 and HDAC is associated with changes in gene expression that leads to growth inhibition and apoptosis (Huang et. al., Carcinogenesis, 34, 1196-1207, 2013). This and other similar studies suggest that the inhibition of both LSD1 and HDAC can exhibit synergism in modulating gene expression and in inducing growth inhibition. MYC is considered a master regulator of human cancers as it plays a key role in modulating the transcription of numerous cancer-related genes.

MYC is considered a master regulator of human cancers as it plays a key role in modulating the transcription of numerous cancer-related genes. Dysregulation in MYC expression is a hallmark of multiple human cancers. MYC amplification is reported in about 15% of all human cancers and is generally associated with poor prognosis and resistance to treatments. Several studies have shown that focal amplification of MYC together with mutation in RB1 and p53 is an important event in the metastatic process of neuroendocrine tumor development. While limited options are available for direct targeting, transcriptional modulation via epigenetic super-enhancer complexes could be an attractive and viable option to target MYC driven neuroendocrine cancers. Therefore, treatment of such MYC amplified cancer is a high unmet need, thus making it an attractive target. While limited options are available for direct targeting, transcriptional modulation via epigenetic super-enhancer complexes appears to be an attractive and viable option to target MYC driven cancers. Epigenetic proteins CREBBP/EP300 and HDACs have been shown to play a significant role in MYC transcription.

Although, there are several chemotherapies and target therapies-based drugs for cancer, an effective cure for cancer remains elusive. Further, development of acquired resistance and disease relapse are major issues that still need to be addressed.

Therefore, there is a need for novel mechanism-based approaches in the treatment of cancer/diseases mediated by LSD1/HDAC proteins, that would have a stronger effect on a signaling pathway and/or affect multiple pathways and mutually exclusive mechanisms in the cells. In this regard, there is still need of dual inhibitors of LSD1/HDAC with better efficacy in treating diseases compared to either treating with LSD1 or HDAC inhibitors individually.

### SUMMARY OF THE DISCLOSURE

In an aspect of the present disclosure, there is provided a compound of Formula (I)

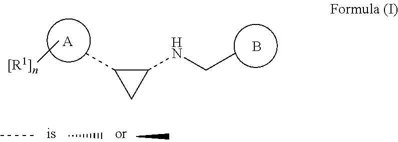

its analogs, tautomeric forms, stereoisomers, enantiomers, diastereomers and pharmaceutically acceptable salts thereof;
wherein,
ring A is selected from a group consisting of C₆₋₁₀aryl, C₁₋₆heteroaryl, and C₂₋₁₀heterocyclyl; wherein, C₁₋₆heteroaryl and C₂₋₁₀heterocyclyl has one or more heteroatoms selected from N, O or S;
R₁ is independently selected from a group consisting of hydrogen, halogen, hydroxy, nitro, cyano, azido, nitroso, oxo, thioxo, —SO₂—, amino, hydrazino, formyl, C₁₋₈alkyl, C₁₋₈haloalkyl, C₁₋₆alkoxy, C₁₋₆haloalkoxy, C₇₋₁₂arylalkoxy, C₃₋₈cycloalkyl, C₃₋₈cycloalkyloxy, C₆₋₁₀aryl, C₂₋₁₀ heterocyclyl, and C₁₋₆heteroaryl; wherein, C₂₋₁₀heterocyclyl, and C₁₋₆heteroaryl has one or more heteroatoms selected from N, O or S;
wherein, C₇₋₁₂arylalkoxy, C₁₋₆alkyl, C₆₋₁₀aryl, C₁₋₆heteroaryl, C₂₋₁₀heterocyclyl, and C₃₋₈ cycloalkyl, is independently optionally substituted with one or more of the groups selected from C₁₋₈ alkyl, C₆₋₁₀aryl, C₁₋₆heteroaryl, C₂₋₁₀heterocyclyl, oxo, C₃₋₈ cycloalkyl, halogen, hydroxy, amino, or cyano;
ring B is

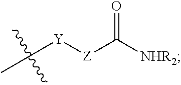

Y is selected from a group consisting of C₁₋₈alkyl, C₂₋₈alkenyl, C₂₋₈alkynyl, C₆₋₁₀aryl, C₁₋₆heteroaryl, C₂₋₁₀heterocyclyl, C₃₋₈cycloalkyl, —CO, and —CO—C₂₋₁₀heterocyclyl; wherein C₁₋₆heteroaryl, C₂₋₁₀heterocyclyl, and —CO—C₂₋₁₀heterocyclyl has one or more heteroatoms selected from N, O or S;
   wherein, C₁₋₆alkyl, C₆₋₁₀aryl, C₁₋₆heteroaryl, C₂₋₁₀heterocyclyl, and C₃₋₈cycloalkyl, is independently optionally substituted with one or more of the groups selected from C₁₋₈ alkyl, oxo, C₃₋₈ cycloalkyl, halogen, hydroxy or cyano;
Z is selected from a group consisting of C₁₋₈alkyl, C₂₋₈alkenyl, C₇₋₁₂-alkylaryl, C₂₋₁₀ heterocyclyl, C₇₋₁₂-arylalkenyl, C₂₋₁₂-alkylheteroaryl, —CO—C₇₋₁₂alkylaryl, —CO—C₇₋₁₂alkenylaryl, —CONR₆—C₁₋₆alkyl, C₆₋₁₀aryl, C₁₋₆heteroaryl, —CO—C₂₋₁₀heterocyclyl, —NR₆—C₆₋₁₀ aryl, —NR₆—C₁₋₆heteroaryl, —O—C₆₋₁₀aryl, —O—C₁₋₆heteroaryl, —CONR₆—C₇₋₁₂alkylaryl, —SO₂—C₆₋₁₀ aryl, —SO₂—C₇₋₁₂alkylaryl, and —NR₆SO₂—C₇₋₁₂alkylaryl; wherein C₂₋₁₀heterocyclyl, C₂₋₁₂-alkylheteroaryl, C₁₋₆heteroaryl, —CO—C₂₋₁₀heterocyclyl, —NR₆—C₁₋₆heteroaryl, and —O—C₁₋₆ heteroaryl has one or more heteroatoms selected from N, O or S;
R₆ is hydrogen or C₁₋₈ alkyl;
R₂ is selected from a group consisting of hydroxy, aniline, C₆₋₁₀aryl, amino C₆₋₁₀aryl, and amino C₁₋₆heteroaryl; wherein amino C₁₋₆heteroaryl has one or more heteroatoms selected from N, O or S;
wherein, aniline, C₆₋₁₀aryl, amino C₆₋₁₀aryl, and amino C₁₋₆heteroaryl, is independently optionally substituted with one or more of the groups selected from amino, C₁₋₈alkyl, halogen, hydroxy or cyano; and n is 1 to 4.

In another aspect of the present disclosure, there is provided a compound of Formula (IA),

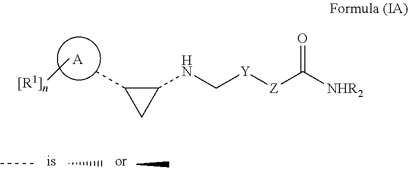

its analogs, tautomeric forms, stereoisomers, enantiomers, diastereomers and pharmaceutically acceptable salts thereof; wherein,
ring A is selected from a group consisting of C₆₋₁₀ aryl, C₁₋₆heteroaryl, and C₂₋₁₀heterocyclyl, wherein, C₁₋₆heteroaryl, and C₂₋₁₀heterocyclyl has one or more heteroatoms selected from N, O or S;
R₁ is independently selected from a group consisting of hydrogen, halogen, oxo, C₁₋₈alkyl, C₆₋₁₀ aryl, C₂₋₁₀heterocyclyl, and C₃₋₆heteroaryl; wherein C₂₋₁₀heterocyclyl and C₃₋₆heteroaryl has one or more heteroatoms selected from N, O or S;
wherein, C₃₋₆heteroaryl, and C₂₋₁₀heterocyclyl, is independently optionally substituted with one or more oxo or C₁₋₈alkyl; Y is selected from a group consisting of C₁₋₆heteroaryl, C₂₋₁₀heterocyclyl, and —CO; wherein C₁6heteroaryl, and C₂₋₁₀heterocyclyl has one or more heteroatoms selected from N, O or S;
wherein, C₁₋₆heteroaryl, and C₂₋₁₀heterocyclyl is independently optionally substituted with one or more oxo or C₁₋₈ alkyl;
Z is selected from a group consisting of C₁₋₈alkyl, C₂₋₈alkenyl, C₇₋₁₂-alkylaryl, C₂₋₁₀ heterocyclyl, —CO—C₇₋₁₂alkylaryl, —CO—C₇₋₁₂alkenylaryl, and C₁₋₆heteroaryl; wherein C₂₋₁₀ heterocyclyl, and C₁₋₆heteroaryl has one or more heteroatoms selected from N, O or S;
R₂ is hydroxy or C₆₋₁₀aryl; wherein, C₆₋₁₀aryl, is substituted with amino; and n is 1 to 4.

In yet another aspect of the present disclosure, there is provided a process of preparation of compounds of Formula (I), its analogs, tautomeric forms, stereoisomers, enantiomers, diastereomers and pharmaceutically acceptable salts thereof, the process comprising the steps of:
Step 1: reacting a compound 1 with an aldehyde or a ketone in a protic solvent to give an intermediate imine followed by reaction with a reducing agent to give an intermediate compound 2 or alternatively reacting compound 1 with an alkyl halide optionally in presence of a base to give intermediate compound 2;
Step 2: hydrolyzing the intermediate compound 2 with an inorganic base to result in the corresponding acid, followed by coupling the acid with the respective amine NH₂R₂ in the presence of an activating agent to yield the compound of Formula (I) or alternatively reacting the intermediate compound 2 with NH₂R₂ and an inorganic base to yield the compound of Formula (I),

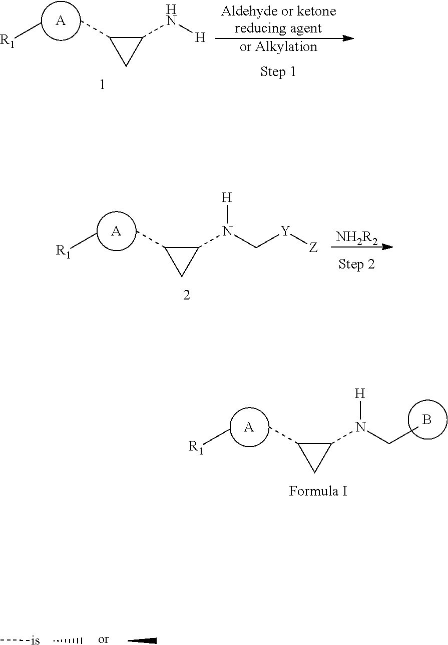

ring A is selected from a group consisting of C₆₋₁₀aryl, C₁₋₆heteroaryl, and C₂₋₁₀heterocyclyl, wherein, C₁₋₆heteroaryl, and C₂₋₁₀heterocyclyl has one or more heteroatoms selected from N, O or S;
R₁ is independently selected from a group consisting of hydrogen, halogen, hydroxy, nitro, cyano, azido, nitroso, oxo, thioxo, —SO₂—, amino, hydrazino, formyl, C₁₋₈alkyl, C₁₋₈haloalkyl, C₁₋₆alkoxy, C₁₋₆haloalkoxy, C₇₋₁₂arylalkoxy, C₃₋₈cycloalkyl, C₃₋₈cycloalkyloxy, C₆₋₁₀aryl, C₂₋₁₀ heterocyclyl, and C₁₋₆heteroaryl; wherein, C₂₋₁₀heterocyclyl, and C₁₋₆heteroaryl has one or more heteroatoms selected from N, O or S;
wherein, C₇₋₁₂arylalkoxy, C₁₋₈alkyl, C₆₋₁₀aryl, C₁₋₆heteroaryl, C₂₋₁₀heterocyclyl, and C₃₋₈ cycloalkyl, is independently optionally substituted with one or more of the groups selected from C₁₋₈ alkyl, C₆₋₁₀aryl, C₁₋₆heteroaryl, C₂₋₁₀heterocyclyl, oxo, C₃₋₈ cycloalkyl, halogen, hydroxy, amino, or cyano;
ring B is

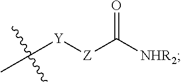

Y is selected from a group consisting of C₁₋₆alkyl, C₂₋₈alkenyl, C₂₋₈alkynyl, C₆₋₁₀aryl, C₁₋₆heteroaryl, C₂₋₁₀heterocyclyl, C₃₋₈cycloalkyl, —CO, and —CO—C₂₋₁₀heterocyclyl; wherein C₁₋₆heteroaryl, C₂₋₁₀heterocyclyl, and —CO—C₂₋₁₀heterocyclyl has one or more heteroatoms selected from N, O or S;
wherein, C₁₋₆alkyl, C₆₋₁₀aryl, C₁₋₆heteroaryl, C₂₋₁₀heterocyclyl, and C₃₋₈cycloalkyl, is independently optionally substituted with one or more of the groups selected from C₁₋₈ alkyl, oxo, C₃₋₈ cycloalkyl, halogen, hydroxy or cyano;
Z is selected from a group consisting of C₁₋₈alkyl, C₂₋₈alkenyl, C₇₋₁₂-alkylaryl, C₂₋₁₀ heterocyclyl, C₇₋₁₂-arylalkenyl, C₂₋₁₂-alkylheteroaryl, —CO—C₇₋₁₂alkylaryl, —CO—C₇₋₁₂alkenylaryl, —CONR₆—C₁₋₈alkyl, C₆₋₁₀aryl, C₁₋₆heteroaryl, —CO—C₂₋₁₀heterocyclyl, —NR₆—C₆₋₁₀ aryl, —NR₆—C₁₋₆heteroaryl, —O—C₆₋₁₀aryl, —O—C₁₋₆heteroaryl, —CONR₆—C₇₋₁₂alkylaryl, —SO₂—C₆₋₁₀ aryl, —SO₂—C₇₋₁₂alkylaryl, and —NR₆SO₂—C₇₋₁₂alkylaryl; wherein C₂₋₁₀heterocyclyl, C₂₋₁₂-alkylheteroaryl, C₁₋₆heteroaryl, —CO—C₂₋₁₀heterocyclyl, —NR₆—C₁₋₆heteroaryl, and —O—C₁₋₆heteroaryl has one or more heteroatoms selected from N, O or S;
R₆ is hydrogen or C₁₋₈ alkyl;
R₂ is selected from a group consisting of hydroxy, aniline, C₆₋₁₀aryl, amino C₆₋₁₀aryl, and amino C₁₋₆heteroaryl; wherein amino C₁₋₆heteroaryl has one or more heteroatoms selected from N, O or S;
wherein, aniline, C₆₋₁₀aryl, amino C₆₋₁₀aryl, and amino C₁₋₆heteroaryl, is independently optionally substituted with one or more of the groups selected from amino, C₁₋₈alkyl, halogen, hydroxy or cyano; and
n is 1 to 4.

In further aspect of the present disclosure, there is provided a pharmaceutical composition comprising a compound of Formula (I) as disclosed herein, and a pharmaceutically acceptable carrier, optionally in combination with one or more other pharmaceutical formulations.

In another aspect of the present disclosure, there is provided a pharmaceutical composition for use in the manufacture of a medicament for inhibiting HDAC enzymes, or LSD1 enzymes or both LSD1 enzymes and HDAC enzymes, or modulating MYC or coREST, in a subject in need thereof, the pharmaceutical composition comprising a compound of Formula (I) as disclosed herein, optionally in combination with one or more other pharmaceutical formulations.

In another aspect of the present disclosure, there is provided a method of treating, preventing, modulating or ameliorating a disease or condition mediated by LSD1 or HDAC or both LSD1 and HDAC in a subject in need thereof, the method comprising: administering to the subject a therapeutically effective amount of a compound of Formula (I) or the pharmaceutical composition as disclosed herein, optionally with other clinically relevant immune modulator agents or other clinically relevant cytotoxic agents or non-cytotoxic agents.

In another aspect of the present disclosure, there is provided a method of treating, preventing, modulating or ameliorating a disease or condition mediated by MYC or coREST in a subject in need thereof, the method comprising: administering to the subject a therapeutically effective amount of a compound of Formula (I) or the pharmaceutical composition as disclosed herein, optionally with other clinically relevant immune modulator agents or other clinically relevant cytotoxic agents or non-cytotoxic agents.

In another aspect of the present disclosure, there is provided a method of inhibiting both LSD1 and HDAC, or transcriptional cofactor in a cell in a subject in need thereof, the method comprising: administering to the subject a therapeutically effective amount of the compound of Formula (I) or the pharmaceutical composition as disclosed herein.

In furthermore aspect of the present disclosure, there is provided a method of treatment of a disease and/or prevention of a condition mediated by both LSD1 and HDAC or a proliferative disorder or cancer, comprising administering to a subject suffering from the condition mediated by both LSD1 and HDAC or the proliferative disorder or cancer, a therapeutically effective amount of the compound of Formula (I) or the pharmaceutical composition as disclosed herein.

In another aspect of the present disclosure, there is provided a method of treatment of a disease and/or prevention of a condition mediated by LSD1 or a proliferative disorder or cancer, comprising administering to a subject suffering from the condition mediated by LSD1 or the proliferative disorder or cancer, a therapeutically effective amount of the compound of Formula (I) or the pharmaceutical composition as disclosed herein.

In one more aspect of the present disclosure, there is provided a method of treatment of a disease and/or prevention of a condition mediated by HDAC or a proliferative disorder or cancer, comprising administering to a subject suffering from the condition mediated by HDAC or the proliferative disorder or cancer, a therapeutically effective amount of the compound of Formula (I) or the pharmaceutical composition as disclosed herein.

In one more aspect of the present disclosure, there is provided a method of treating a subject with a tumor, the method comprising: administering to the subject, a therapeutically effective amount of the compound of Formula (I) or the pharmaceutical composition as disclosed herein.

In further more aspect of the present disclosure, there is provided a method of treating a subject with cancer, the method comprising: administering to the subject a therapeutically effective amount of the compound of Formula (I) or the pharmaceutical composition as disclosed herein.

These and other features, aspects, and advantages of the present subject matter will become better understood with reference to the following description and the appended claims. This summary is provided to introduce a selection of concepts in a simplified form. This summary is not intended to identify key features or essential features of the disclosure, nor is it intended to be used to limit the scope of the claimed subject matter.

### BRIEF DESCRIPTION OF THE DRAWINGS

In addition to the features described herein, additional features and variations will be readily apparent from the following descriptions of the drawings and exemplary embodiments. It is to be understood that these drawings depict typical embodiments, and are not intended to be limiting in scope.

FIG. 1 illustrates a western-blot results depicting Example 1, ORY-1001 (LSD1 inhibitor), and ACY-1215 (HDAC inhibitor) effects on c-Myc modulation in erythroleukemia cells, in accordance with an implementation of the present disclosure.

FIG. 2 illustrates a line graph depicting effects of Example 1 and docetaxel (standard of care) on average tumor volume on prostate cancer xenograft model, in accordance with an implementation of the present disclosure.

### DETAILED DESCRIPTION

Those skilled in the art will be aware that the present disclosure is subject to variations and modifications other than those specifically described. It is to be understood that the present disclosure includes all such variations and modifications. The disclosure also includes all such steps, features, compositions and compounds referred to or indicated in this specification, individually or collectively, and any and all combinations of any or more of such steps or features.

### Definitions

For convenience, before further description of the present disclosure, certain terms employed in the specification, and examples are collected here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The term “or” means “and/or” unless stated otherwise.

The terms “comprise” and “comprising” are used in the inclusive, open sense, meaning that additional elements may be included. Throughout this specification, unless the context requires otherwise the word “comprise”, and variations, such as “comprises” and “comprising”, will be understood to imply the inclusion of a stated element or step or group of element or steps but not the exclusion of any other element or step or group of element or steps.

The term “compound(s)” comprises the compounds disclosed in the present disclosure.

The term “including” is used to mean “including but not limited to”. “Including” and “including but not limited to” are used interchangeably.

In the structural formulae given herein and throughout the present disclosure, the following terms have been indicated meaning, unless specifically stated otherwise.

Furthermore, the compound of Formula (I) can be its analogs, tautomeric forms, stereoisomers, enantiomers, diastereomers, geometrical isomers or pharmaceutically acceptable salts and compositions.

The compounds described herein may contain one or more chiral centers and/or double bonds and therefore, may exist as stereoisomers, such as double-bond isomers (i.e., geometric isomers), regioisomers, enantiomers or diastereomers. Accordingly, the chemical structures depicted herein encompass all possible enantiomers and stereoisomers of the illustrated or identified compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the person skilled in the art. The compounds may also exist in several tautomeric forms including the enol form, the keto form and mixtures thereof. Accordingly, the chemical structures depicted herein encompass all possible tautomeric forms of the illustrated or identified compounds. It is also understood that some isomeric form such as diastereomers, enantiomers and geometrical isomers can be separated by physical and/or chemical methods and by those skilled in the art.

The term “enantiomers” refers to the two stereoisomers of the compound of Formula (I) that are non-superimposable mirror images of each other. Enantiomeric excess refers to the absolute difference between each enantiomer. Enantiomeric excess refers to chiral compounds having more of one enantiomer than the other.

According to the present disclosure, the compounds provided herein, includes all of the corresponding enantiomers and stereoisomers, that is, the pure form of the stereoisomers, in terms of geometrical isomer, enantiomer, or diastereomer, and the mixture of enantiomeric and stereoisomeric form of said compounds. Further, the mixture of enantiomeric and stereoisomeric form can be resolved into their pure component by the methods known in the art, such as chiral-phase gas chromatography, chiral-phase high performance liquid chromatography, crystallization, using chiral derivatizing agents etc. Also, the pure enantiomers and stereoisomers can be obtained from reagents that are in the form of pure enantiomers and stereoisomers by known asymmetric synthetic methods.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents, for example, include those described herein above. The permissible substituents can be one or more and the same or different for appropriate organic compounds. For purposes of this disclosure, the heteroatoms such as nitrogen may have hydrogen substituents, and/or any permissible substituents of organic compounds described herein which satisfy the valences of the heteroatoms.

Furthermore, the term “substituted” or “optionally substituted” refers to replacement of one or more hydrogen radicals in a given structure with a radical of a specified substituent including, but not limited to: halo, oxo, sulpho, sulphido, alkyl, alkenyl, alkynyl, aryl, heterocyclyl, thiol, alkylthio, arylthio, alkylthioalkyl, arylthioalkyl, alkylsulfonyl, alkyl sulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, cycloalkyl, heteroaryl, and aliphatic. It is understood that the substituent may be further substituted.

The term “alkyl” refers to straight or branched aliphatic hydrocarbon groups having the specified number of carbon atoms, which are attached to the rest of the molecule by a single atom, which may be optionally substituted by one or more substituents. Preferred alkyl groups include, without limitation, methyl, ethyl, n-propyl, isopropyl, butyl, isobutyl, t-butyl, pentyl, hexyl, heptyl, octyl and the like.

The term “aryl” is optionally substituted monocyclic, bicyclic or polycyclic aromatic hydrocarbon ring system of about 6 to 10 carbon atoms. Examples of C₆-C₁₀ aryl groups include, but are not limited to phenyl, or naphthyl, Aryl group can be optionally substituted with one or more suitable groups.

The term “heterocyclyl” refers to a heterocyclic ring radical which may be optionally substituted by one or more substituents. The heterocyclyl ring radical may be attached to the main structure at any heteroatom or carbon atom that results in the creation of a stable structure.

Furthermore, the term “heterocyclyl” refers to a stable 2 to 10 membered rings radical, which consists of carbon atoms and from one to five heteroatoms selected from nitrogen, phosphorus, oxygen and sulfur. For the purpose of this disclosure the heterocyclic ring radical may be monocyclic, bicyclic or tricyclic ring systems, and the nitrogen, phosphorus, carbon, or sulfur atoms in the heterocyclic ring radical may be optionally oxidized to various oxidation states. In addition, the nitrogen atom may be optionally quaternized; and the ring radical may be partially or fully saturated. Preferred heterocyclyl groups include, without limitation, azetidinyl, acridinyl, benzodioxolyl, benzodioxanyl, benzofuranyl, carbazolyl, cinnolinyl, dioxolanyl, indolizinyl, naphthyridinyl, perhydroazepinyl, phenazinyl, phenothiazinyl, phenoxazinyl, phthalazinyl, pyridyl, pteridinyl, purinyl, quinazolinyl, qunioxalinyl, quinolinyl, isoquinolinyl, tetrazolyl, imidazolyl, tetrahydroisoquinolinyl, piperidinyl, piperazinyl, homopiperazinyl, 2-oxoazepinyl, azepinyl, pyrrolyl, 4-piperidonyl, pyrrolidinyl, pyrazinyl, pyrimidinyl, pyridazinyl, oxazolyl, oxazolinyl, triazolyl, indanyl, isoxazolyl, isoxazolidinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzooxazolyl, thienyl, morpholinyl, thiomorpholinyl, thiamorpholinyl sulfoxide, furyl, tetrahydrofuryl, tetrahydropyranyl, chromanyl, and isochromanyl.

The term “heteroaryl” alone or in combination with other term(s) means a completely unsaturated ring system containing a total of 5 to 14 ring atoms having at least one carbon atom. At least one of the ring atoms is a heteroatom (i.e., oxygen, nitrogen, or sulfur), with the remaining ring atoms/groups being independently selected from carbon, oxygen, nitrogen or sulfur. A heteroaryl may be a single-ring (monocyclic) or multiple rings (bicyclic, tricyclic or polycyclic) fused together or linked covalently. Preferably, “heteroaryl” is a 5- to 6-membered ring. The rings may contain from 1 to 4 additional heteroatoms selected from N, O and S, wherein the N atom is optionally quarternized. Any suitable ring position of the heteroaryl moiety may be covalently linked to the defined chemical structure. Examples of “heteroaryl” include but are not limited to furanyl, thienyl, pyrrolyl, pyrazolyl, imidazolyl, oxazolyl, cinnolinyl, isoxazolyl, thiazolyl, isothiazolyl, 1H-tetrazolyl, oxadiazolyl, triazolyl, pyridyl, 3-fluoropyridyl, pyrimidinyl, pyrazinyl, pyridazinyl, benzoxazolyl, benzisoxazolyl; benzothiazolyl, benzofuranyl, benzothienyl, benzotriazinyl, phthalazinyl, thianthrene, dibenzofuranyl, dibenzothienyl, benzimidazolyl, indolyl, isoindolyl, indazolyl, quinolinyl, isoquinolinyl, quinazolinyl, quinoxalinyl, purinyl, pteridinyl, 9H-carbazolyl, a-carbolinyl, indolizinyl, benzoisothiazolyl, benzoxazolyl, pyrrolopyridyl, furopyridinyl, purinyl, benzothiadiazolyl, benzooxadiazolyl, benzotriazolyl, benzotriadiazolyl, carbazolyl, dibenzothienyl, acridinyl and the like. Heteroaryl group may be optionally further substituted.

The term “heteroatom” as used herein designates a sulfur, a nitrogen or an oxygen atom.

The term “alkylheteroaryl” refers to alkyl attached to heteroaryl group and may be optionally substituted. For the purpose of the present disclosure, the alkylheteroaryl refers to compounds with carbon atoms ranging between 2 to 12, which includes the alkyl group with 1 to 6 carbon atoms.

The term “fused heterocyclyl” refers to monocyclic or polycyclic ring, polycyclic ring system refers to a ring system containing 2 or more rings, preferably bicyclic or tricyclic rings, in which rings can be fused, bridged or spiro rings or any combinations thereof. A fused ring as used herein means that the two rings are linked to each other through two adjacent ring atoms common to both rings. The fused ring can contain 1-4 heteroatoms independently selected from N, O, and S. The rings can be either fused by nitrogen or —CH— group.

The term “bridged ring” as used herein means that a ring comprises a linker group (C(Rq)₂)p-linking together any two non-adjacent carbon or nitrogen atoms of the ring, where p is 1 or 2 and each independently is hydrogen or C₁₋₄ alkyl.

The term “cycloalkyl” refers to non-aromatic mono or polycyclic ring system of about 3 to 8 carbon atoms, which may be optionally substituted by one or more substituents. The polycyclic ring denotes hydrocarbon systems containing two or more ring systems with one or more ring carbon atoms in common i.e. a spiro, fused or bridged structures. Preferred cycloalkyl groups include, without limitation, cyclopropyl, cyclobutyl, cyclopentyl, cyclohexyl, cyclooctanyl, bridged cyclic groups or spirobicyclic groups e.g., spiro [4.4]non-2-yl and the like.

The term “alkoxy” refers to an alkyl group attached via an oxygen linkage to the rest of the molecule, which may be optionally substituted by one or more substituents. Preferred alkoxy groups include, without limitation, —OCH₃, —OC₂H₅ and the like.

The term “arylalkoxy” refers to a group having alkyl attached to the alkoxy group. For the purpose of the present disclosure, the arylalkoxy group has 7 to 12 carbon atoms.

The term “halo” or “halogen” alone or in combination with other term(s) means fluorine, chlorine, bromine or iodine.

The term “haloalkoxy” refers to alkoxy with one or more halogen atoms substituted for hydrogen in the alkoxy group. In the present disclosure, haloalkoxy refers to compounds with 1 to 8 carbon atoms and examples of haloalkoxy includes but not limited to —OCH₂F, —OCHF₂, —OCF₃, —OC₂H4F, and the like.

The term “cycloalkyloxy” refers to cycloalkyl group attached via an oxygen linkage in the cycloalkyl molecule, which may be optionally substituted by one or more substituents.

The term “haloalkyl” refers to alkyl with one or more halogen atoms. In the present disclosure, the term haloalkyl refers to compounds with 1 to 8 carbon atoms and examples of haloalkyl includes but not limited to —CH₂F, —CHF₂, —CF₃, —C₂H4F and the like.

The term “alkenyl” refers to an aliphatic hydrocarbon group containing a carbon-carbon double bond and which may be straight or branched chain having about 2 to 8 carbon atoms, which may be optionally substituted by one or more substituents. Preferred alkenyl groups include, without limitation, ethenyl, 1-propenyl, 2-propenyl, iso-propenyl, 2-methyl-1-propenyl, 1-butenyl, 2-butenyl and the like.

The term “alkynyl” refers to a straight or branched hydrocarbyl radicals having at least one carbon-carbon triple bond and having in the range of 2-8 carbon atoms, which may be optionally substituted by one or more substituents. Preferred alkynyl groups include, without limitation, ethynyl, propynyl, butynyl and the like.

The term “alkylaryl” refers to an aryl group directly bonded to an alkyl group, which may be optionally substituted by one or more substituents. Preferred alkylaryl groups include, without limitation, —CH₂-phenyl-, —C₂H₄-phenyl-, C₃H₆-phenyl-, and the like.

The term “alkenylaryl” refers to an alkenyl group directly bonded to an aryl group, which may be optionally substituted by one or more substituents. Preferred alkenylaryl groups include, without limitation, —CH═CH-phenyl, —CH₂—CH═CH-phenyl and the like.

The term “arylalkenyl” refers to an aryl group directly bonded to an alkenyl group, which may be optionally substituted by one or more substituents. Preferred arylalkenyl groups include, without limitation, —C₆H₅—CH═CH—, —C₆H₅—CH═CH—CH₂— and the like.

The term “arylalkynyl” refers to an aryl group directly bonded to an alkynyl group, which may be optionally substituted by one or more substituents. Preferred arylalkenyl groups include, without limitation, —C₆H₅-ethynyl-, —C₆H₅-propynyl-, and the like.

The term “—CO-alkylaryl” refers to a carbonyl group directly attached to an alkylaryl group which may be optionally substituted by one or more substituents. Preferred “—CO-alkylaryl groups include, without limitations, —CO—CH₂-phenyl-, —CO—C₂H₄-phenyl- and the like.

The term “—CO-alkenylaryl” refers to a carbonyl group directly attached to an alkenylaryl group which may be optionally substituted by one or more substituents. Preferred “—CO-alkenylaryl” groups include, without limitations, —CO—CH═CH-phenyl-, —CO—CH₂—CH═CH-phenyl- and the like.

The term “—CO-heterocyclyl” refers to a carbonyl group directly attached through the heteroatom or carbon atom of a heterocyclyl group which may be optionally substituted by one or more substituents. Preferred “—CO-heterocyclyl” groups include, without limitations, —CO-piperazinyl, —CO—N-piperdinyl (implies attachment is through the nitrogen of piperdinyl group), —CO—C-piperidinyl (implies the attachment is through the carbon of piperdinyl group) and the like.

The term “cyano” refers to a —CN group.

The term “oxo” refers to a ═O group.

The term “amino” refers to a —NH₂ group.

The term “hydroxy” or “hydroxyl” refers to a —OH group.

The term “thioxo” refers to =S group.

The term “hydrazino” refers to a —NH—NH₂ group.

The term “formyl” refers to a

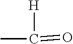

group.

The term “nitro” refers to —NO₂ group.

The term “nitroso” refers to —N═O group.

The term “azido” refers to —N₃ group.

The term “one or more” used herein refers to one to many, may be one to ten. In the present disclosure, the term “one or more of the groups selected from” refers to selecting one group or more groups from the list. This would also refer to selecting one group or more of same group, or more of different groups from the list or combination of the groups.

It is understood that included in the family of compounds of Formula (I) are isomeric forms including diastereomers, enantiomers, tautomers and geometrical isomers in “E” or “Z” configurational isomer or a mixture of ‘E’ and ‘Z’ isomers. It is also understood that some isomeric form such as diastereomers, enantiomers and geometrical isomers can be separated by physical and/or chemical methods and by those skilled in the art.

Compounds disclosed herein may exist as single stereoisomers, and or mixtures of enantiomers and/or diastereomers. All such single stereoisomers, and mixtures thereof are intended to be within the scope of the subject matter described.

Compounds disclosed herein include isotopes of hydrogen, carbon, oxygen, fluorine, chlorine, iodine and sulfur which can be incorporated into the compounds, such as not limited to ²H (D), ¹¹C, ¹³C, ¹⁵N, ¹⁸F, ³⁵S, ³⁶Cl and ¹²⁵I. Compounds of this disclosure where in atoms were isotopically labeled can be used in metabolic studies, kinetic studies and imaging techniques such as positron emission tomography used in understanding the tissue distribution of the drugs. Compounds of the disclosure where hydrogen is replaced with deuterium may improve the metabolic stability and pharmacokinetics properties of the drug such as in vivo half-life. Compounds of the disclosure where isotopically labeled ¹⁸F can be useful as PET imaging studies.

As used herein, the term “composition” is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

As used herein, the term “pharmaceutical composition” refers to a composition(s) containing a therapeutically effective amount of at least one compound of formula (I) or their pharmaceutically acceptable salt; and a conventional pharmaceutically acceptable carrier.

The pharmaceutical composition(s) usually contain(s) about 1% to 99%, for example, about 5% to 75%, or from about 10% to about 30% by weight of the compound of formula (I) or pharmaceutically acceptable salts thereof. The amount of the compound of formula (I) or pharmaceutically acceptable salts thereof in the pharmaceutical composition(s) can range from about 1 mg to about 1000 mg or from about 2.5 mg to about 500 mg or from about 5 mg to about 250 mg or in any range falling within the broader range of 1 mg to 1000 mg or higher or lower than the afore mentioned range.

The term “other pharmaceutical formulation(s)” refers to other active pharmaceutical ingredients or formulation that can work in combination with the compound of Formula (I) or with the pharmaceutical composition of the present disclosure.

As used herein, the term “treat”, “treating” and “treatment” refer to any treatment of a disease in a mammal, including: (a) Inhibiting the disease, i.e., slowing or arresting the development of clinical symptoms; and/or (b) relieving the disease, i.e., causing the regression of clinical symptoms and/or (c) alleviating or abrogating a disease and/or its attendant symptoms.

As used herein, the term “prevent”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

As used herein, the term “subject” that may be interchangeable with ‘patient’, refers to an animal, preferably a mammal, and most preferably a human.

As used herein, the term “therapeutically effective amount” refers to that amount of a compound of Formula (I), its analogs, tautomeric forms, stereoisomers, enantiomers, diastereomers or pharmaceutically acceptable salts thereof or a composition comprising the compound of Formula (I), its analogs, tautomeric forms, stereoisomers, enantiomers, diastereomers or pharmaceutically acceptable salts thereof; effective in producing the desired therapeutic response in a particular patient suffering from a diseases or disorder, in particular their use in diseases or disorder associated with cancer. Particularly, the term “therapeutically effective amount” includes the amount of the compound of Formula (I) or a pharmaceutically acceptable salt or a stereoisomer thereof; when administered, that induces a positive modification in the disease or disorder to be treated or is sufficient to prevent development of, or alleviate to some extent, one or more of the symptoms of the disease or disorder being treated in a subject in need thereof. In respect of the therapeutic amount of the compound, the amount of the compound used for the treatment of a subject is low enough to avoid undue or severe side effects, within the scope of sound medical judgment can also be considered. The therapeutically effective amount of the compound or composition will be varied with the particular condition being treated, the severity of the condition being treated or prevented, the duration of the treatment, the nature of concurrent therapy, the age and physical condition of the end user, the specific compound or composition employed the particular pharmaceutically acceptable carrier utilized.

The phrase “pharmaceutically acceptable” refers to compounds or compositions that are physiologically tolerable and do not typically produce allergic or similar untoward reaction, generally safe, non-toxic and neither biologically nor otherwise undesirable and includes that which is acceptable for veterinary as well as human pharmaceutical use, including but not limited to gastric upset or dizziness when administered to subjects.

Pharmaceutically acceptable salts forming part of this disclosure include salts derived from inorganic bases such as like Li, Na, K, Ca, Mg, Fe, Cu, Zn and Mn and ammonium, substituted ammonium salts, aluminum salts and the like; salts of organic bases such as N, N′-diacetylethylenediamine, glucamine, triethylamine, choline, dicyclohexylamine, benzylamine, trialkylamine, thiamine, guanidine, diethanolamine, α-phenylethylamine, piperidine, morpholine, pyridine, hydroxyethylpyrrolidine, hydroxyethylpiperidine and the like, salts also include amino acid salts such as glycine, alanine, cystine, cysteine, lysine, arginine, phenylalanine, guanidine etc. Salts may include acid addition salts where appropriate which are sulphates, nitrates, phosphates, perchlorates, borates, hydrohalides, acetates, tartrates, maleates, fumarates, citrates, succinates, lactates, mesylates, trifluoroacetates, acetates, besylates, propionates, mandelates, hydrobromides, hydrochlorides, palmoates, methanesulphonates, tosylates, benzoates, salicylates, hydroxynaphthoates, benzenesulfonates, ascorbates, glycerophosphates, ketoglutarates and the like.

The terms “histone deacetylase” and “HDAC” are intended to refer to any one of a family of enzymes that remove acetyl groups from the F-amino groups of lysine residues at the N-terminus of a histone or tubulin. Unless otherwise indicated by context, the term “histone” is meant to refer to any histone protein, including H1, H2A, H2B, H3, H4 and H5, from any species. Human HDAC proteins or gene products include but are not limited to, HDAC-1, HDAC-2, HDAC-3, HDAC-4, HDAC-5, HDAC-6, HDAC-7, HDAC-8, HDAC-9, HDAC-10 and HDAC-11. The histone deacetylase can also be derived from a protozoal or fungal source.

The term “histone deacetylase inhibitor” or “inhibitor of histone deacetylase” is used to identify a compound, which is capable of interacting with a histone deacetylase and inhibiting its activity, more particularly its enzymatic activity. Inhibiting histone deacetylase enzymatic activity means reducing the ability of a histone deacetylase to remove an acetyl group from a histone or tubulin. Preferably, such inhibition is specific, i.e. the histone deacetylase inhibitor reduces the ability of histone deacetylase to remove an acetyl group from a histone or tubulin at a concentration that is lower than the concentration of the inhibitor that is required to produce some other, unrelated biological effect.

The term “lysine demethylase inhibitor” or “inhibitor of lysine demethylase” is used to identify a compound, which is capable of interacting with a histone demethylase and inhibiting its activity, more particularly its enzymatic activity. Inhibiting histone demethylase enzymatic activity means reducing the ability of a histone demethylase to remove a methyl group from a histone. Inhibitor of histone demethylase involves removal of either mono methyl or dimethyl or trimethyl group from histones. Preferably, such inhibition is specific, i.e. the histone demethylase inhibitor reduces the ability of histone demethylase to remove a methyl group from a histone at a concentration that is lower than the concentration of the inhibitor that is required to produce some other, unrelated biological effect.

The term ‘Dual inhibitor of LSD-1/HDAC’ is capable of removing acetyl group from histones or tubulin and methyl group from histones. These inhibitors are capable of inhibiting more than one HDAC isozyme and all those isozymes are covered in addition to inhibiting LSD-1 activity

The term dual inhibitor LSD1/HDAC6 is used to identify a compound which is capable of interacting selectively with HDAC6 enzymes in addition to having enzymatic interactions for LSD-1. Dual inhibitor of LSD-1/HDAC6 is capable of removing acetyl group from tubulin and methyl group from histones.

The term dual inhibitor LSD1/HDAC1 is used to identify a compound which is capable of interacting selectively with HDAC1 enzymes in addition to having enzymatic interactions for LSD-1. Dual inhibitor of LSD-1/HDAC1 is capable of removing acetyl group from histones and methyl group from histones.

The term dual inhibitor LSD1/HDAC8 is used to identify a compound which is capable of interacting selectively with HDAC8 enzymes in addition to having enzymatic interactions for LSD-1. Dual inhibitor of LSD-1/HDAC8 is capable of removing acetyl group from histones and methyl group from histones.

The term “MYC” refers to a polymer of amino acid residues disclosed in NCBI Accession Number NP002458.2, and functional homologs, analogs, or fragments thereof; or an amino acid sequence that is at least 40% to 100% identical to the sequence of NCBI Accession Numbers NP002458.2 or to a nucleic acid sequence that encodes a nucleotide sequence of at least 120 nucleotides that is at least 60% to 100% identical or homologous identical to sequences of NCBI Accession Number NM_002467. The compounds of the present disclosure modulates MYC, which can be further defined as downregulation of MYC RNA and/or degradation of MYC protein. The compounds of the present disclosure also inhibits the transcriptional regulator coREST.

The term “coREST” refers to a newly identified protein, which functions as a corepressor for REST. CoREST is a repressor protein; mutations that disrupt CoREST's binding to REST also interfere with REST repression.

The term “REST” refers to RE1-Silencing Transcription factor (REST), also known as Neuron-Restrictive Silencer Factor (NRSF), which is a transcriptional repressor which acts via epigenetic remodeling to silence target genes.

The term “immune checkpoint inhibitors agents” or “immune modulators agents” are used to identify any agents or inhibitors that block certain proteins made by some types of immune system cells, such as T cells, and some cancer cells. These proteins help keep immune responses in check and can keep T cells from killing cancer cells. When these proteins are blocked, the “brakes” on the immune system are released and T cells are able to kill cancer cells better. The immune checkpoint inhibitors include inhibitors against immune checkpoint molecules such as CD27, CD28, CD40, CD 122, CD96, CD73, CD47, OX40, GITR, CSF1R, JAK, PI3K delta, PI3K gamma, TAM arginase, CD137 (also known as 4-1B), ICOS, A2AR, B7-H3, B7-H4, BTLA, CTLA-4, LAG3, TIM3, VISTA, PD-1, PD-L1 and PD-L2. The terms “immune modulators agents” and “immune checkpoint inhibitors” are used interchangeably throughout the present invention.

The term “cytotoxic agents” or “cytotoxic inhibitors” is used to identify any agents or drugs which are capable of killing cells including cancer cells. These agents or inhibitors may stop cancer cells from growing and dividing and may cause tumors to shrink in size.

The term “non-cytotoxic agents” or “non-cytotoxic inhibitors” is used to identify any agents or inhibitors are which does not directly kill cells, but instead affects cellular transport and metabolic functions to ultimately produce cell death.

A term once described, the same meaning applies for it, throughout the patent.

As discussed in the background, it can be observed that there is a tremendous need for novel mechanism-based approaches in the treatment of cancer/diseases mediated by LSD1/HDAC enzymes. It has been surprisingly determined that the specific enantiomeric forms of a compounds of Formula (I) have great effect on biological activity as compared to their racemic forms. The present disclosure thus provides enantiomeric forms of compounds of Formula (I) thereof, that results in unexpected properties. The present disclosure substantially provides a pure preparation of higher activity specific enantiomeric forms of compounds of Formula (I) that has surprisingly higher activity than the racemic forms of said compounds and other related compounds. Additionally, the compounds of the present disclosure had been found to modulate MYC gene and/or MYC protein, which are useful in treating various diseases.

In an implementation of the present disclosure, there is provided a compound of Formula (I),

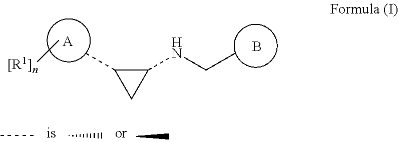

its analogs, tautomeric forms, stereoisomers, enantiomers, diastereomers and pharmaceutically acceptable salts thereof;
ring A is selected from a group consisting of C₆₋₁₀aryl, C₁₋₆heteroaryl, and C₂₋₁₀heterocyclyl, wherein, C₁₋₆heteroaryl, and C₂₋₁₀heterocyclyl has one or more heteroatoms selected from N, O or S;
R₁ is independently selected from a group consisting of hydrogen, halogen, hydroxy, nitro, cyano, azido, nitroso, oxo, thioxo, —SO₂—, amino, hydrazino, formyl, C₁₋₈alkyl, C₁₋₈haloalkyl, C₁₋₆alkoxy, C₁₋₈haloalkoxy, C₇₋₁₂arylalkoxy, C₃₋₈cycloalkyl, C₃₋₈cycloalkyloxy, C₆₋₁₀aryl, C₂₋₁₀heterocyclyl, and C₁₋₆heteroaryl; wherein, C₂₋₁₀heterocyclyl, and C₁₋₆heteroaryl has one or more heteroatoms selected from N, O or S;
wherein, C₇₋₁₂arylalkoxy, C₁₋₈alkyl, C₆₋₁₀aryl, C₁₋₆heteroaryl, C₂₋₁₀heterocyclyl, and C₃₋₈cycloalkyl, is independently optionally substituted with one or more of the groups selected from C₁₋₈ alkyl, C₆₋₁₀aryl, C₁₋₆heteroaryl, C₂₋₁₀heterocyclyl, oxo, C₃₋₈ cycloalkyl, halogen, hydroxy, amino, or cyano;
ring B is

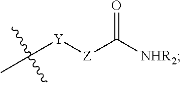

Y is selected from a group consisting of C₁₋₈alkyl, C₂₋₈alkenyl, C₂₋₈alkynyl, C₆₋₁₀aryl, C₁₋₆heteroaryl, C₂₋₁₀heterocyclyl, C₃₋₈cycloalkyl, —CO, and —CO—C₂₋₁₀heterocyclyl; wherein C₁₋₆heteroaryl, C₂₋₁₀heterocyclyl, and —CO—C₂₋₁₀heterocyclyl has one or more heteroatoms selected from N, O or S;
   wherein, C₁₋₈alkyl, C₆₋₁₀aryl, C₁₋₆heteroaryl, C₂₋₁₀heterocyclyl, and C₃₋₈cycloalkyl, is independently optionally substituted with one or more of the groups selected from C₁₋₆ alkyl, oxo, C₃₋₈ cycloalkyl, halogen, hydroxy or cyano;
Z is selected from a group consisting of C₁₋₈alkyl, C₂₋₈alkenyl, C₇₋₁₂-alkylaryl, C₂₋₁₀ heterocyclyl, C₇₋₁₂-arylalkenyl, C₂₋₁₂-alkylheteroaryl, —CO—C₇₋₁₂alkylaryl, —CO—C₇₋₁₂alkenylaryl, —CONR₆—C₁₋₈alkyl, C₆₋₁₀aryl, C₁₋₆heteroaryl, —CO—C₂₋₁₀heterocyclyl, —NR₆—C₆₋₁₀ aryl, —NR₆—C₁₋₆heteroaryl, —O—C₆₋₁₀aryl, —O—C₁₋₆heteroaryl, —CONR₆—C₇₋₁₂alkylaryl, —SO₂—C₆₋₁₀aryl, —SO₂—C₇₋₁₂alkylaryl, and —NR₆SO₂—C₇₋₁₂alkylaryl; wherein C₂₋₁₀heterocyclyl, C₂₋₁₂-alkylheteroaryl, C₁₋₆heteroaryl, —CO—C₂₋₁₀heterocyclyl, —NR₆—C₁₋₆heteroaryl, and —O—C₁₋₆heteroaryl has one or more heteroatoms selected from N, O or S;
R₆ is hydrogen or C₁₋₈ alkyl;
R₂ is selected from a group consisting of hydroxy, aniline, C₆₋₁₀aryl, amino C₆₋₁₀aryl, and amino C₁₋₆heteroaryl; wherein amino C₁₋₆heteroaryl has one or more heteroatoms selected from N, O or S;
wherein, aniline, C₆₋₁₀aryl, amino C₆₋₁₀aryl, and amino C₁₋₆heteroaryl, is independently optionally substituted with one or more of the groups selected from amino, C₁₋₈alkyl, halogen, hydroxy or cyano; and
n is 1 to 4.

In an implementation of the present disclosure, there is provided a compound of Formula (IA),

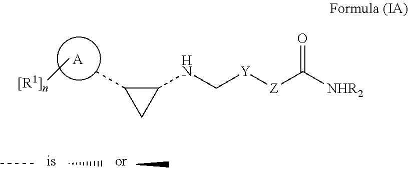

its analogs, tautomeric forms, stereoisomers, enantiomers, diastereomers and pharmaceutically acceptable salts thereof;
wherein,
ring A is selected from a group consisting of C₆₋₁₀aryl, C₁₋₆heteroaryl, and C₂₋₁₀heterocyclyl, wherein, C₁₋₆heteroaryl, and C₂₋₁₀heterocyclyl has one or more heteroatoms selected from N, O or S;
R₁ is independently selected from a group consisting of hydrogen, halogen, oxo, C₁₋₈alkyl, C₆₋₁₀ aryl, C₂₋₁₀heterocyclyl, and C₃₋₆heteroaryl; wherein C₂₋₁₀heterocyclyl and C₃₋₆heteroaryl has one or more heteroatoms selected from N, O or S;
wherein, C₃₋₆heteroaryl, and C₂₋₁₀heterocyclyl, is independently optionally substituted with one or more oxo or C₁₋₈alkyl;
Y is selected from a group consisting of C₁₋₆heteroaryl, C₂₋₁₀heterocyclyl, and —CO; wherein C₁₋₆heteroaryl, and C₂₋₁₀heterocyclyl has one or more heteroatoms selected from N, O or S wherein, C₁₋₆heteroaryl, and C₂₋₁₀heterocyclyl is independently optionally substituted with one or more oxo or C₁₋₈ alkyl;
Z is selected from a group consisting of C₁₋₈alkyl, C₂₋₈alkenyl, C₇₋₁₂-alkylaryl, C₂₋₁₀ heterocyclyl, —CO—C₇₋₁₂alkylaryl, —CO—C₇₋₁₂alkenylaryl, and C₁₋₆heteroaryl; wherein C₂₋₁₀ heterocyclyl, and C₁₋₆heteroaryl has one or more heteroatoms selected from N, O or S;
R₂ is hydroxy or C₆₋₁₀aryl; wherein, C₆₋₁₀aryl, is substituted with amino; and n is 1 to 4.

In an implementation of the present disclosure, there is provided a compound of Formula (I) as disclosed herein, wherein, ring A is selected from a group consisting of C₆₋₁₀aryl, C₁₋₆heteroaryl and C₂₋₁₀heterocyclyl, wherein, C₁₋₆heteroaryl, and C₂₋₁₀ heterocyclyl has one or more heteroatoms selected from N, O or S; R₁ is independently selected from a group consisting of hydrogen, halogen, hydroxy, nitro, oxo, amino, hydrazino, C₁₋₈alkyl, C₁₋₈alkoxy, C₃₋₈cycloalkyl, C₆₋₁₀aryl, C₂₋₁₀heterocyclyl, and C₁₋₆heteroaryl; wherein, C₁₋₈alkyl, C₆₋₁₀aryl, C₁₋₆heteroaryl, C₂₋₁₀heterocyclyl, and C₃₋₈cycloalkyl, is independently optionally substituted with one or more of the groups selected from C₁₋₈ alkyl, C₆₋₁₀aryl, halogen or hydroxy; Y is selected from a group consisting of C₁₋₈alkyl, C₆₋₁₀aryl, C₁₋₆heteroaryl, C₂₋₁₀heterocyclyl, C₃₋₈cycloalkyl, —CO, and —CO—C₂₋₁₀heterocyclyl; wherein, C₁₋₆alkyl, C₆₋₁₀aryl, C₁₋₆heteroaryl, C₂₋₁₀heterocyclyl, C₃₋₈cycloalkyl, is independently optionally substituted with one or more of the groups selected from C₁₋₈ alkyl, oxo, halogen, hydroxy or cyano; Z is selected from a group consisting of C₁₋₈alkyl, C₂₋₈alkenyl, C₇₋₁₂-alkylaryl, C₂₋₁₀ heterocyclyl, C₇₋₁₂-arylalkenyl, C₂₋₁₂-alkylheteroaryl, —CO—C₇₋₁₂alkylaryl, —CO—C₇₋₁₂alkenylaryl, —CONR₆—C₁₋₆alkyl, C₆₋₁₀aryl, C₁₋₆heteroaryl, —CO—C₂₋₁₀heterocyclyl; R₂ is selected from a group consisting of hydroxy, aniline, C₆₋₁₀aryl, amino C₆₋₁₀aryl, and amino C₁₋₆heteroaryl; wherein aniline, C₆₋₁₀aryl, amino C₆₋₁₀aryl, and amino C₁₋₆heteroaryl, is independently optionally substituted with one or more of the groups selected from amino, C₁₋₆alkyl, halogen, hydroxy or cyano; and n is 1 to 4.

In an implementation of the present disclosure, there is provided a compound of Formula (I) as disclosed herein, wherein, ring A is selected from a group consisting of C₆₋₈aryl, C₁₋₆heteroaryl and C₂₋₁₀heterocyclyl, wherein, C₁₋₆heteroaryl, and C₂₋₁₀ heterocyclyl has one or more heteroatoms selected from N, O or S; R₁ is independently selected from a group consisting of hydrogen, halogen, hydroxy, nitro, oxo, amino, hydrazino, C₁₋₈alkyl, C₁₋₈alkoxy, C₃₋₈cycloalkyl, C₆₋₈aryl, C₂₋₁₀heterocyclyl, and C₁₋₆heteroaryl; wherein, C₁₋₆alkyl, C₆₋₈aryl, C₁₋₆heteroaryl, C₂₋₁₀heterocyclyl, and C₃₋₈cycloalkyl, is independently optionally substituted with one or more of the groups selected from C₁₋₆ alkyl, C₆₋₈aryl, halogen or hydroxy; Y is selected from a group consisting of C₁₋₈alkyl, C₆₋₈aryl, C₁₋₆heteroaryl, C₂₋₁₀ heterocyclyl, C₃₋₈cycloalkyl, —CO, and —CO—C₂₋₁₀heterocyclyl; wherein, C₁₋₈alkyl, C₆₋₈aryl, C₁₋₆heteroaryl, C₂₋₁₀heterocyclyl, and C₃₋₈cycloalkyl, is independently optionally substituted with one or more of the groups selected from C₁₋₆ alkyl, oxo, halogen, hydroxy or cyano; Z is selected from a group consisting of C₁₋₈alkyl, C₂₋₈alkenyl, C₇₋₁₂-alkylaryl, C₂₋₁₀heterocyclyl, C₇₋₁₂-arylalkenyl, C₂₋₁₂-alkylheteroaryl, —CO—C₇₋₁₂alkylaryl, —CO—C₇₋₁₂alkenylaryl, —CONR₆—C₁₋₈alkyl, C₆₋₈aryl, C₁₋₆heteroaryl, and —CO—C₂₋₁₀heterocyclyl; R₂ is selected from a group consisting of hydroxy, aniline, C₆₋₈aryl, amino C₆₋₈aryl, and amino C₁₋₆heteroaryl; wherein aniline, C₆₋₈aryl, amino C₆₋₈aryl, and amino C₁₋₆heteroaryl, is optionally substituted with one or more of the groups selected from amino, C₁₋₆alkyl, halogen, hydroxy or cyano; and n is 1 to 4.

In an implementation of the present disclosure, there is provided a compound of Formula (I) as disclosed herein, wherein, ring A is selected from a group consisting of C₆₋₁₀ aryl, C₁₋₆heteroaryl, and C₂₋₁₀heterocyclyl, wherein, C₁₋₆heteroaryl, and C₂₋₁₀heterocyclyl has one or more heteroatoms selected from N, O or S; R₁ is independently selected from a group consisting of hydrogen, halogen, oxo, C₁₋₈alkyl, C₆₋₁₀aryl, C₂₋₁₀heterocyclyl, and C₃. 6heteroaryl; wherein C₂₋₁₀heterocyclyl and C₃₋₆heteroaryl has one or more heteroatoms selected from N, O or S; wherein, C₃₋₆heteroaryl, and C₂₋₁₀heterocyclyl, is independently optionally substituted with one or more oxo or C₁₋₈alkyl; Y is selected from a group consisting of C₁₋₆heteroaryl, C₂₋₁₀heterocyclyl, and —CO; wherein, C₁₋₆heteroaryl, and C₂₋₁₀heterocyclyl is independently optionally substituted with one or more oxo or C₁₋₈ alkyl; Z is selected from a group consisting of C₁₋₈alkyl, C₂₋₈alkenyl, C₇₋₁₂-alkylaryl, C₂₋₁₀heterocyclyl, —CO—C₇₋₁₂alkylaryl, —CO—C₇₋₁₂alkenylaryl, and C₁₋₆heteroaryl; R₂ is hydroxy or C₆₋₁₀aryl; wherein, C₆₋₁₀ aryl, is substituted with amino; and n is 1 to 4.

In an implementation of the present disclosure, there is provided a compound of Formula (I) as disclosed herein, wherein ring A is selected from a group consisting of C₆₋₈aryl, C₁₋₆heteroaryl, and C₂₋₁₀heterocyclyl, wherein, C₁₋₆heteroaryl, and C₂₋₁₀heterocyclyl has one or more heteroatoms selected from N, O or S; R₁ is independently selected from a group consisting of hydrogen, halogen, oxo, C₁₋₈alkyl, C₆₋₈aryl, C₂₋₁₀heterocyclyl, and C₃₋₆heteroaryl; wherein C₂₋₁₀heterocyclyl and C₃₋₆heteroaryl has one or more heteroatoms selected from N, O or S; wherein, C₃₋₆heteroaryl, and C₂₋₁₀heterocyclyl, is independently optionally substituted with one or more oxo or C₁₋₈alkyl; Z is selected from a group consisting of C₁₋₈alkyl, C₂₋₈alkenyl, C₇₋₁₂-alkylaryl, C₂₋₁₀heterocyclyl, —CO—C₇₋₁₂alkylaryl, —CO—C₇₋₁₂alkenylaryl, and C₁₋₆ heteroaryl; R₂ is hydroxy or C₆₋₈aryl; wherein, C₆₋₈aryl, is substituted with amino; and n is 1 to 4.

In an implementation of the present disclosure, there is provided a compound of Formula (I) or Formula (IA) as disclosed herein, wherein ring A is selected from a group consisting of C₆₋₈ aryl, C₃₋₆heteroaryl, and C₅₋₉heterocyclyl, wherein, C₃₋₆heteroaryl, and C₅₋₉ heterocyclyl has one or more heteroatoms selected from N, O or S; R₁ is independently selected from a group consisting of hydrogen, halogen, oxo, C₁₋₄alkyl, C₆₋₈aryl, C₅₋₉ heterocyclyl, and C₃₋₆heteroaryl; wherein C₅₋₉heterocyclyl and C₃₋₅heteroaryl has one or more heteroatoms selected from N, O or S; wherein, C₅₋₉heterocyclyl and C₃₋₅heteroaryl is independently optionally substituted with one or more oxo or C₁₋₄alkyl; Y is selected from a group consisting of C₂₋₅heteroaryl, and C₅₋₉heterocyclyl; wherein C₂₋₅heteroaryl, and C₅₋₉ heterocyclyl has one or more heteroatoms selected from N, O or S, wherein, C₂₋₅heteroaryl, and C₅₋₉heterocyclyl is independently optionally substituted with one or more oxo or C₁₋₄ alkyl; Z is selected from a group consisting of C₁₋₄alkyl, C₇₋₉-alkylaryl, and —CO—C₇₋₁₀alkenylaryl; R₂ is hydroxy or C₆₋₈aryl; wherein, C₆₋₈aryl is substituted with amino; and n is 1 to 4.

In an implementation of the present disclosure, there is provided a compound of Formula (I) as disclosed herein, wherein, the compound of Formula (I) acts as LSD1 inhibitor or HDAC inhibitor or both LSD1/HDAC inhibitor.

In an implementation of the present disclosure, there is provided a compound of Formula (I) as disclosed herein, wherein, the compound of Formula (I) acts as an epigenetic modulating agent.

In an implementation of the present disclosure, there is provided a compound of Formula (I) as disclosed herein, wherein, the compound of Formula (I) modulates MYC.

In an implementation of the present disclosure, there is provided a compound of Formula (I) as disclosed herein, wherein, the compound of Formula (I) regulates transcriptional cofactors.

In an implementation of the present disclosure, there is provided a compound of Formula (I) as disclosed herein, wherein, the compound of Formula (I) blocks neuroendocrine transdifferentiation.

In an implementation of the present disclosure, there is provided a compound of Formula (I) as disclosed herein, wherein, the compound of Formula (I) is selected from the group consisting of:
1. N-hydroxy-4-((4-((((1R,2S)-2-phenylcyclopropyl)amino)methyl)-1H-pyrazol-1-yl)methyl)benzamide hydrobromide;
2. N-hydroxy-4-((4-((((1S,2R)-2-phenylcyclopropyl)amino)methyl)-1H-pyrazol-1-yl)methyl)benzamide;
3. N-hydroxy-4-(3-(4-((((1R,2S)-2-(1-phenyl-1H-pyrazol-4-yl)cyclopropyl)amino)methyl)piperidin-1-yl)propyl)benzamide;
4. N-hydroxy-4-(3-(4-((((1S,2R)-2-(1-phenyl-1H-pyrazol-4-yl)cyclopropyl)amino)methyl)piperidin-1-yl)propyl)benzamide;
5. 4-((E)-3-(3-((((1R,2S)-2-(4-fluorophenyl)cyclopropyl)amino)methyl)azetidin-1-yl)-3-oxoprop-1-en-1-yl)-N-hydroxybenzamide;
6. 4-((E)-3-(3-((((1S,2R)-2-(4-fluorophenyl)cyclopropyl)amino)methyl)azetidin-1-yl)-3-oxoprop-1-en-1-yl)-N-hydroxybenzamide;
7. N-hydroxy-4-(3-(4-((((1R,2S)-2-(4-(pyrimidin-5-yl)phenyl)cyclo propyl)amino)methyl)piperidin-1-yl)propyl)benzamide;
8. N-hydroxy-4-(3-(4-((((1S,2R)-2-(4-(pyrimidin-5-yl)phenyl)cyclo propyl)amino)methyl)piperidin-1-yl)propyl)benzamide;
9. N-hydroxy-4-(3-(4-((((1R,2S)-2-(1,3,3-trimethyl-2-oxoindolin-5-yl)cyclopropyl)amino)methyl)piperidin-1-yl)propyl)benzamide;
10. N-hydroxy-4-(3-(4-((((1S,2R)-2-(1,3,3-trimethyl-2-oxoindolin-5-yl)cyclopropyl)amino)methyl)piperidin-1-yl)propyl)benzamide;
11. N-hydroxy-4-((4-((((1R,2S)-2-phenylcyclopropyl)amino)methyl)piperidin-1-yl)methyl)benzamide;
12. N-hydroxy-4-((4-((((1S,2R)-2-phenylcyclopropyl)amino)methyl)piperidin-1-yl)methyl)benzamide;
13. N-hydroxy-4-(3-(4-((((1R,2S)-2-phenylcyclopropyl)amino)methyl)piperidin-1-yl)propyl)benzamide;
14. N-hydroxy-4-(3-(4-((((1S,2R)-2-phenylcyclopropyl)amino)methyl)piperidin-1-yl)propyl)benzamide;
15. N-(2-aminophenyl)-4-(3-(4-((((1R,2S)-2-(4-fluorophenyl)cyclopropyl) amino)methyl)piperidin-1-yl)propyl)benzamide;
16. N-(2-aminophenyl)-4-(3-(4-((((1S,2R)-2-(4-fluorophenyl)cyclopropyl) amino)methyl)piperidin-1-yl)propyl)benzamide;
17. N-(2-aminophenyl)-4-(3-(4-((((1R,2S)-2-(1-isopropyl-1H-pyrazol-4-yl)cyclopropyl)amino)methyl)piperidin-1-yl)propyl)benzamide;
18. N-(2-aminophenyl)-4-(3-(4-((((1S,2R)-2-(1-isopropyl-1H-pyrazol-4-yl)cyclopropyl)amino)methyl)piperidin-1-yl)propyl)benzamide;
19. N-(2-aminophenyl)-4-((4-((((1R,2S)-2-(4-(1-methyl-6-oxo-1,6-dihydropyridin-3-yl)phenyl)cyclopropyl)amino)methyl)piperidin-1-yl)methyl)benzamide;
20. N-(2-aminophenyl)-4-((4-((((1S,2R)-2-(4-(1-methyl-6-oxo-1,6-dihydropyridin-3-yl)phenyl)cyclopropyl)amino)methyl)piperidin-1-yl)methyl)benzamide;
21. N-(2-aminophenyl)-4-((4-((((1R,2S)-2-(4-fluorophenyl)cyclopropyl) amino)methyl)-1H-1,2,3-triazol-1-yl)methyl)benzamide;
22. N-(2-aminophenyl)-4-((4-((((1S,2R)-2-(4-fluorophenyl)cyclopropyl) amino)methyl)-1H-1,2,3-triazol-1-yl)methyl)benzamide;
23. N-(2-aminophenyl)-4-(3-(4-((((1R,2S)-2-(1-phenyl-1H-pyrazol-4-yl)cyclopropyl)amino)methyl)piperidin-1-yl)propyl)benzamide;
24. N-(2-aminophenyl)-4-(3-(4-((((1S,2R)-2-(1-phenyl-1H-pyrazol-4-yl)cyclopropyl)amino)methyl)piperidin-1-yl)propyl)benzamide;
25. N-(2-aminophenyl)-4-(3-(4-((((1R,2S)-2-(3,4-difluorophenyl) cyclopropyl)amino)methyl)piperidin-1-yl)propyl)benzamide;
26. N-(2-aminophenyl)-4-(3-(4-((((1S,2R)-2-(3,4-difluorophenyl) cyclopropyl)amino)methyl)piperidin-1-yl)propyl)benzamide;
27. N-(2-aminophenyl)-4-((4-((((1R,2S)-2-(4-(3,5-dimethylisoxazol-4-yl)phenyl)cyclopropyl)amino)methyl)piperidin-1-yl)methyl)benzamide;
28. N-(2-aminophenyl)-4-((4-((((1S,2R)-2-(4-(3,5-dimethylisoxazol-4-yl)phenyl)cyclopropyl)amino)methyl)piperidin-1-yl)methyl)benzamide;
29. N-(2-aminophenyl)-4-((4-((((1R,2S)-2-phenylcyclopropyl)amino)methyl)-1H-1,2,3-triazol-1-yl)methyl)benzamide; and
30. N-(2-aminophenyl)-4-((4-((((1R,2S)-2-phenylcyclopropyl)amino)methyl)-1H-pyrazol-1-yl)methyl)benzamide.

In an implementation of the present disclosure, there is provided a process of preparation of compounds of Formula (I) as disclosed herein, the process comprising the steps of:
Step 1: reacting a compound 1 with an aldehyde or ketone in a protic solvent to give an intermediate imine followed by reaction with a reducing agent to give an intermediate compound 2 or alternatively reacting compound 1 with alkyl halide optionally in the presence of base to give intermediate compound 2;
Step 2: hydrolyzing the intermediate compound 2 with an inorganic base to result in the corresponding acid, followed by coupling the acid with the respective amine NH₂R₂ in the presence of activating agent to yield the compound of Formula (I) or alternatively reacting the intermediate compound 2 with NH₂R₂ and an inorganic base to yield the compound of Formula (I),

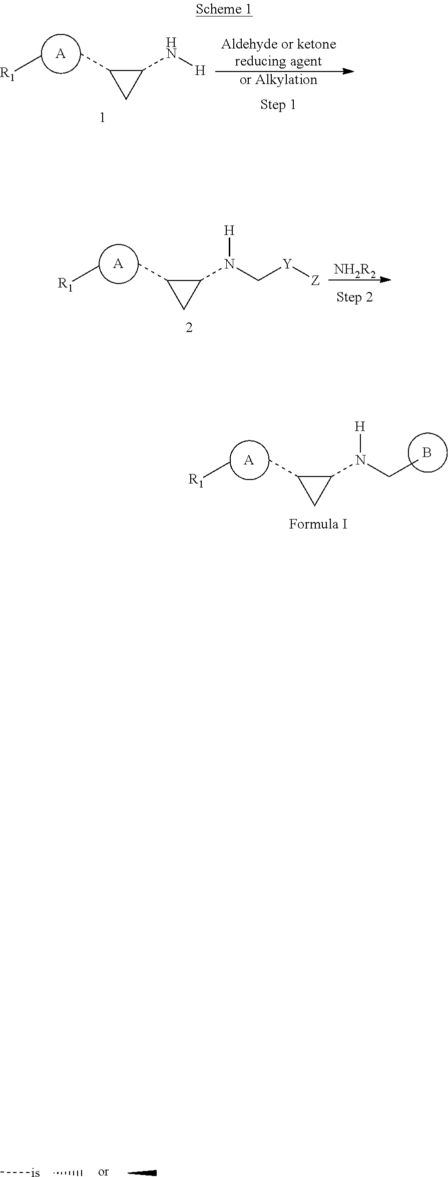

ring A is selected from a group consisting of C₆₋₁₀aryl, C₁₋₆heteroaryl, and C₂₋₁₀heterocyclyl, wherein, C₁₋₆heteroaryl, and C₂₋₁₀heterocyclyl has one or more heteroatoms selected from N, O or S;
R₁ is independently selected from a group consisting of hydrogen, halogen, hydroxy, nitro, cyano, azido, nitroso, oxo, thioxo, —SO₂—, amino, hydrazino, formyl, C₁₋₈alkyl, C₁₋₈haloalkyl, C₁₋₆alkoxy, C₁₋₆haloalkoxy, C₇₋₁₂arylalkoxy, C₃₋₈cycloalkyl, C₃₋₈cycloalkyloxy, C₆₋₁₀aryl, C₂₋₁₀ heterocyclyl, and C₁₋₆heteroaryl; wherein, C₂₋₁₀heterocyclyl, and C₁₋₆heteroaryl has one or more heteroatoms selected from N, O or S;
wherein, C₇₋₁₂arylalkoxy, C₁₋₈alkyl, C₆₋₁₀aryl, C₁₋₆heteroaryl, C₂₋₁₀heterocyclyl, and C₃₋₈cycloalkyl, is independently optionally substituted with one or more of the groups selected from C₁₋₈ alkyl, C₆₋₁₀aryl, C₁₋₆heteroaryl, C₂₋₁₀heterocyclyl, oxo, C₃₋₈ cycloalkyl, halogen, hydroxy, amino, or cyano;
ring B is

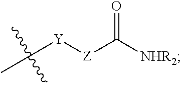

Y is selected from a group consisting of C₁₋₈alkyl, C₂₋₈alkenyl, C₂₋₈alkynyl, C₆₋₁₀aryl, C₁₋₆heteroaryl, C₂₋₁₀heterocyclyl, C₃₋₈cycloalkyl, —CO, and —CO—C₂₋₁₀heterocyclyl; wherein C₁₋₆ heteroaryl, C₂₋₁₀heterocyclyl, and —CO—C₂₋₁₀heterocyclyl has one or more heteroatoms selected from N, O or S;
wherein, C₁₋₆alkyl, C₆₋₁₀aryl, C₁₋₆heteroaryl, C₂₋₁₀heterocyclyl, and C₃₋₈cycloalkyl, is independently optionally substituted with one or more of the groups selected from C₁₋₈ alkyl, oxo, C₃₋₈ cycloalkyl, halogen, hydroxy or cyano;
Z is selected from a group consisting of C₁₋₈alkyl, C₂₋₈alkenyl, C₇₋₁₂-alkylaryl, C₂₋₁₀ heterocyclyl, C₇₋₁₂-arylalkenyl, C₂₋₁₂-alkylheteroaryl, —CO—C₇₋₁₂alkylaryl, —CO—C₇₋₁₂alkenylaryl, —CONR₆—C₁₋₆alkyl, C₆₋₁₀aryl, C₁₋₆heteroaryl, —CO—C₂₋₁₀heterocyclyl, —NR₆—C₆₋₁₀ aryl, —NR₆—C₁₋₆heteroaryl, —O—C₆₋₁₀aryl, —O—C₁₋₆heteroaryl, —CONR₆—C₇₋₁₂alkylaryl, —SO₂—C₆₋₁₀ aryl, —SO₂—C₇₋₁₂alkylaryl, and —NR₆SO₂—C₇₋₁₂alkylaryl; wherein C₂₋₁₀heterocyclyl, C₂₋₁₂-alkylheteroaryl, C₁₋₆heteroaryl, —CO—C₂₋₁₀heterocyclyl, —NR₆—C₁₋₆heteroaryl, and —O—C₁₋₆heteroaryl has one or more heteroatoms selected from N, O or S;
R₆ is hydrogen or C₁₋₈ alkyl;
R₂ is selected from a group consisting of hydroxy, aniline, C₆₋₁₀aryl, amino C₆₋₁₀aryl, and amino C₁₋₆heteroaryl;
   wherein, aniline, C₆₋₁₀aryl, amino C₆₋₁₀aryl, and amino C₁₋₆heteroaryl, is independently optionally substituted with one or more of the groups selected from amino, C₁₋₈ alkyl, halogen, hydroxy or cyano; and
n is 1 to 4.

In an implementation of the present disclosure, there is provided a process of preparation of compounds of Formula (I) as disclosed herein, wherein, the activating agent is selected from EDCI. HCl (1(3-dimethylaminopropyl)-3-ethylcarbodiimide hydrochloride), HOBt (1-hydroxybenzotriazole) or (1-propylphosphonic anhydride)T3P/triethylamine; the protic solvent is selected from methanol, ethanol, water, or combinations thereof; and the reducing agent is selected from sodium borohydride, lithium borohydride, aluminum borohydride, or combinations thereof.

In an implementation of the present disclosure, there is provided a process of preparation of compounds of Formula (I) as disclosed herein, wherein, the base is selected from triethylamine, N, N-diisopropylethylamine, potassium carbonate, pyridine, or combinations thereof; and the inorganic base is selected from potassium carbonate, sodium carbonate, cesium carbonate, or combinations thereof.

In an implementation of the present disclosure, there is provided a process of preparation of compounds of Formula (I) as disclosed herein, wherein, the protic solvent is selected from methanol, ethanol, water or combinations thereof; and the reducing agent is selected from sodium borohydride, lithium borohydride, aluminum borohydride or combinations thereof. In another embodiment of the present disclosure, the protic solvent is methanol and the reducing agent is sodium borohydride.

In an implementation of the present disclosure, there is provided a pharmaceutical composition comprising a compound of Formula (I) as disclosed herein and a pharmaceutically acceptable carrier.

In an implementation of the present disclosure, there is provided a pharmaceutical composition comprising a compound of Formula (I) as disclosed herein and a pharmaceutically acceptable carrier, optionally in combination with one or more other pharmaceutically acceptable carrier.

In an implementation of the present disclosure, there is provided a pharmaceutical composition as disclosed herein, wherein the composition is in the form selected from a group consisting of a tablet, capsule, powder, syrup, solution, aerosol and suspension.

In an implementation of the present disclosure, there is provided a pharmaceutical composition comprising a compound of Formula (I) as disclosed herein, wherein the compound of Formula (I) has enantiomeric excess of higher than 60%, preferably higher than 80%.

In an implementation of the present disclosure, there is provided a pharmaceutical composition comprising a compound of Formula (I) as disclosed herein, and a pharmaceutically acceptable carrier, optionally in combination with one or more other pharmaceutical formulations for use in the manufacture of a medicament for inhibiting HDAC enzymes or LSD1 enzymes, or both HDAC enzymes and LSD1 enzymes, or modulating MYC or coREST in a subject in need thereof.

In an implementation of the present disclosure, there is provided a pharmaceutical composition comprising a compound of Formula (I) as disclosed herein, and a pharmaceutically acceptable carrier, optionally in combination with one or more other pharmaceutical formulations for use in the manufacture of a medicament for inhibiting HDAC enzymes or LSD1 enzymes, or both HDAC enzymes and LSD1 enzymes in a subject in need thereof.

In an implementation of the present disclosure, there is provided a pharmaceutical composition comprising a compound of Formula (I) as disclosed herein, and a pharmaceutically acceptable carrier, optionally in combination with one or more other pharmaceutical formulations for use in the manufacture of a medicament for inhibiting both HDAC enzymes and LSD1 enzymes.

In an implementation of the present disclosure, there is provided a pharmaceutical composition comprising a compound of Formula (I) as disclosed herein, and a pharmaceutically acceptable carrier, optionally in combination with one or more other pharmaceutical formulations for use in the manufacture of a medicament for inhibiting HDAC enzymes.

In an implementation of the present disclosure, there is provided a pharmaceutical composition comprising a compound of Formula (I) as disclosed herein, and a pharmaceutically acceptable carrier, optionally in combination with one or more other pharmaceutical formulations for use in the manufacture of a medicament for inhibiting LSD1 enzymes.

In an implementation of the present disclosure, there is provided a pharmaceutical composition comprising a compound of Formula (I) as disclosed herein, and a pharmaceutically acceptable carrier, optionally in combination with one or more other pharmaceutical formulations for use in the manufacture of a medicament for modulating MYC or coREST complex.

In an implementation of the present disclosure, there is provided a pharmaceutical composition comprising a compound of Formula (I) or a pharmaceutically acceptable salt thereof as disclosed herein, and a pharmaceutically acceptable carrier, optionally in combination with one or more other pharmaceutical formulations for use in the manufacture of a medicament for modulating coREST complex.

In an implementation of the present disclosure, there is provided a pharmaceutical composition comprising a compound of Formula (I) or a pharmaceutically acceptable salt thereof as disclosed herein, and a pharmaceutically acceptable carrier, optionally in combination with one or more other pharmaceutical formulations for use in the manufacture of a medicament for modulating MYC.

In an implementation of the present disclosure, there is provided a compound of Formula (I) thereof as disclosed herein, for use in manufacture of a medicament for inhibiting HDAC enzymes, LSD1 enzymes, or both HDAC enzymes and LSD1 enzymes, or for modulating MYC or coREST in a cell for treating a disease or a condition.

In an implementation of the present disclosure, there is provided a compound of Formula (I)s as disclosed herein, for use in manufacture of a medicament for inhibiting HDAC enzymes, LSD1 enzymes, or both HDAC enzymes and LSD1 enzymes, in a cell for treating a disease or a condition.

In an implementation of the present disclosure, there is provided a compound of Formula (I) as disclosed herein, for use in manufacture of a medicament for inhibiting both HDAC enzymes and LSD1 enzymes, in a cell for treating a disease or a condition.

In an implementation of the present disclosure, there is provided a compound of Formula (I) as disclosed herein, for use in manufacture of a medicament for inhibiting HDAC enzymes, in a cell for treating a disease or a condition.

In an implementation of the present disclosure, there is provided a compound of Formula (I) as disclosed herein, for use in manufacture of a medicament for inhibiting LSD1 enzymes, in a cell for treating a disease or a condition.

In an implementation of the present disclosure, there is provided a compound of Formula (I) as disclosed herein, for use in manufacture of a medicament for modulating MYC or coREST complex, in a cell for treating a disease or a condition.

In an implementation of the present disclosure, there is provided a compound of Formula (I) as disclosed herein, for use in manufacture of a medicament for modulating MYC, in a cell for treating a disease or a condition.

In an implementation of the present disclosure, there is provided a compound of Formula (I) as disclosed herein, for use in manufacture of a medicament for modulating coREST complex, in a cell for treating a disease or a condition.

In an implementation of the present disclosure, there is provided a compound of Formula (I) or a pharmaceutical composition as disclosed herein, for use in the manufacture of a medicament for inhibiting HDAC enzymes, LSD1 enzymes, or both HDAC enzymes and LSD1 enzymes, in a cell for treating breast cancer, prostate cancer, pancreatic cancer, gastric cancer, lung cancer, colon cancer, rectal cancer, esophagus cancer, duodenal cancer, tongue cancer, pharyngeal cancer, brain tumor, neurinoma, clear cell carcinoma, non-small cell lung cancer, small cell lung cancer, liver cancer, liver metastasis, kidney cancer, bile duct cancer, uterine body cancer, cervical cancer, ovarian cancer, urinary bladder, skin cancer, hemangioma, malignant lymphoma, malignant melanoma, thyroid cancer, bone tumor, vascular fibroma, glioblastoma, sarcoma, neuroendocrine tumors, retinoblastoma, penile cancer, pediatric solid cancer, renal cell carcinoma, lymphoma, myeloma and leukemia including, for example acute myelogenous leukemia (AML), chronic myelogenous leukemia (CML), chronic neutrophilic leukemia, chronic eosinophilic leukemia, chronic lymphocytic leukemia (CLL), acute lymphoblastic leukemia (ALL), hairy cell leukemia, cutaneous T-cell lymphoma (CTCL), multiple myeloma (MM), Myeloproliferative neoplasms (MPN), polycythemia vera (PV), myeloproliferative dysplastic syndrome, essential thrombocythemia (ET), essential thrombocytosis and myelofibrosis (MF), chronic myelogenous leukemia (CML), chronic neutrophilic leukemia (CNL), chronic eosinophilic leukemia (CEL), cancers with specific mutations in the SWI/SNF complex including but not limited to ARID1A, ARID1B, SMARCA4, SMARCA2, cancers with mutations in specific oncogenes, including but not limited to EGFR, KRAS, RET, cancers with differential expression of certain genes including but not limited to LAPTM5, RNASE6, IL-16, APOBEC3 and TYROBP, depression, Alzheimer's disease, Huntington disease, Parkinson's disease, Amyotrophic lateral sclerosis, Dementia with lewy bodies, Frontotemporal dementia or MYC amplified tumor.

In an implementation of the present disclosure, there is provided a compound of Formula (I) as disclosed herein, for use in the manufacture of a medicament for inhibiting HDAC enzymes, LSD1 enzymes, or both HDAC enzymes and LSD1 enzymes, in a cell for treating multiple myeloma (MM), myeloproliferative neoplasms (MPN), polycythemia vera (PV), myeloproliferative dysplastic syndrome, essential thrombocythemia (ET), essential thrombocytosis or myelofibrosis (MF).

In an implementation of the present disclosure, there is provided use of the compounds of Formula (I) or the pharmaceutical composition as disclosed herein, for treatment of a condition mediated by LSD1 or HDAC or both LSD1 and HDAC; treatment and/or prevention of a proliferative disorder or cancer; or treatment of cancer together with other clinically relevant cytotoxic agents or non-cytotoxic agents.

In an implementation of the present disclosure, there is provided a method of inhibiting both LSD1 and HDAC, or transcriptional cofactor in a cell in a subject in need thereof, the method comprising: administering to the subject a therapeutically effective amount of the compound of formula (I) or the pharmaceutical composition as disclosed herein.

In an implementation of the present disclosure, there is provided a method of inhibiting transcriptional cofactor in a cell in a subject in need thereof, the method comprising: administering to the subject a therapeutically effective amount of the compound of Formula (I) or the pharmaceutical composition as disclosed herein.

In an implementation of the present disclosure, there is provided a method of inhibiting both LSD1 and HDAC in a cell in a subject in need thereof, the method comprising: administering to the subject a therapeutically effective amount of the compound of Formula (I) or the pharmaceutical composition as disclosed herein.

In an implementation of the present disclosure, there is provided a method of treatment and/or prevention of a disease or a condition mediated by both LSD1 and HDAC or a proliferative disorder or cancer, comprising administering to a subject suffering from the disease or condition mediated by both LSD1 and HDAC or the proliferative disorder or cancer, a therapeutically effective amount of the compound of Formula (I) or pharmaceutical composition as disclosed herein.

In an implementation of the present disclosure, there is provided a method of treatment and/or prevention of a disease or a condition comprising administering to a subject in need thereof an effective amount of compound of Formula (I) or pharmaceutical composition as disclosed herein with other clinically relevant immune modulator agents to a subject in need of thereof.

In an implementation of the present disclosure, there is provided a method of treatment and/or prevention of a disease or a condition comprising administering to a subject in need thereof an effective amount of compound of Formula (I) or pharmaceutical composition as disclosed herein with other clinically relevant immune modulator agents; wherein other clinically relevant immune modulator agents are selected from CD27, CD28, CD40, CD 122, CD96, CD73, CD47, OX40, GITR, CSF1R, JAK, PI3K delta, PI3K gamma, TAM arginase, CD137 (also known as 4-1BB), ICOS, A2AR, B7-H3, B7-H4, BTLA, CTLA-4, LAG3, TIM3, VISTA, PD-1, PD-L1 and PD-L2.

In an implementation of the present disclosure, there is provided a method of treatment of a disease and/or prevention of a condition comprising administering to a subject in need thereof an effective amount of compound of Formula (I) or pharmaceutical composition as disclosed herein with other clinically relevant cytotoxic agents or non-cytotoxic agents to a subject in need thereof.

In an implementation of the present disclosure, there is provided a method of treatment and/or prevention of a disease or a condition mediated by MYC RNA & MYC protein, comprising administering to a subject suffering from the disease or condition mediated by MYC, a therapeutically effective amount of the compound of Formula (I) or the pharmaceutical composition as disclosed herein.

In an implementation of the present disclosure, there is provided a method of treatment and/or prevention of a disease or a condition comprising administering to a subject in need thereof an effective amount of compound of Formula (I) or pharmaceutical composition as disclosed herein, wherein the disease or condition is selected from breast cancer, prostate cancer, pancreatic cancer, gastric cancer, lung cancer, colon cancer, rectal cancer, esophagus cancer, duodenal cancer, tongue cancer, pharyngeal cancer, brain tumor, neurinoma, clear cell carcinoma, non-small cell lung cancer, small cell lung cancer, liver cancer, liver metastasis, kidney cancer, bile duct cancer, uterine body cancer, cervical cancer, ovarian cancer, urinary bladder, skin cancer, hemangioma, malignant lymphoma, malignant melanoma, thyroid cancer, bone tumor, vascular fibroma, glioblastoma, sarcoma, neuroendocrine tumors, retinoblastoma, penile cancer, pediatric solid cancer, renal cell carcinoma, lymphoma, myeloma and leukemia (including, for example acute myelogenous leukemia (AML), chronic myelogenous leukemia (CML), chronic neutrophilic leukemia, chronic eosinophilic leukemia, chronic lymphocytic leukemia (CLL), acute lymphoblastic leukemia (ALL), hairy cell leukemia, cutaneous T-cell lymphoma (CTCL), multiple myeloma (MM), Myeloproliferative neoplasms (MPN), polycythemia vera (PV), myeloproliferative dysplastic syndrome, essential thrombocythemia (ET), essential thrombocytosis and myelofibrosis (MF), chronic myelogenous leukemia (CML), chronic neutrophilic leukemia (CNL), chronic eosinophilic leukemia (CEL), cancers with specific mutations in the SWI/SNF complex including but not limited to ARID1A, ARID1B, SMARCA4, SMARCA2, or cancers with mutations in specific oncogenes, EGFR, KRAS, RET, cancers with differential expression of certain genes including, LAPTM5, RNASE6, IL-16, APOBEC3 and TYROBP, depression, Alzheimer's disease, Huntington disease, Parkinson's disease, Amyotrophic lateral sclerosis, Dementia with lewy bodies, Frontotemporal dementia or MYC amplified tumor.

In an implementation of the present disclosure, there is provided a method of treatment and/or prevention of a disease or a condition comprising administering to a subject in need thereof an effective amount of compound of Formula (I) or pharmaceutical composition as disclosed herein, wherein the disease or condition is selected from multiple myeloma (MM), Myeloproliferative neoplasms (MPN), polycythemia vera (PV), myeloproliferative dysplastic syndrome, essential thrombocythemia (ET), essential thrombocytosis or myelofibrosis (MF).

In an implementation of the present disclosure, there is provided use of the compound of Formula (I) or the pharmaceutical composition as disclosed herein, in manufacturing a medicament for treatment and/or prevention of a disease or a condition.

In an implementation of the present disclosure, there is provided use of the compound of Formula (I) or the pharmaceutical composition as disclosed herein, for treatment of a disease or a condition mediated by LSD1 or HDAC or both LSD1 and HDAC, or MYC or coREST, treatment and/or prevention of a proliferative disorder or cancer, or treatment of cancer together with other clinically relevant cytotoxic agents or non-cytotoxic agents.

In an implementation of the present disclosure, there is provided use of the compound of Formula (I) or the pharmaceutical composition as disclosed herein, for treatment of a disease or a condition mediated by LSD1 or HDAC or both LSD1 and HDAC, treatment and/or prevention of a proliferative disorder or cancer or treatment of cancer together with other clinically relevant cytotoxic agents or non-cytotoxic agents.

In an implementation of the present disclosure, there is provided use of the compound of Formula (I) or the pharmaceutical composition as disclosed herein, for treatment of a disease or a condition mediated by MYC or coREST, treatment and/or prevention of a proliferative disorder or cancer; or treatment of cancer together with other clinically relevant cytotoxic agents or non-cytotoxic agents.

In an implementation of the present disclosure, there is provided use of the compound of Formula (I) or the pharmaceutical composition as disclosed herein, for treatment and/or prevention of a disease or a condition mediated by MYC.

In an implementation of the present disclosure, there is provided a method of treating and/or preventing a neurodegenerative disease or disorder comprising administering, to a patient in need of treatment, a therapeutically effectively amount of a composition comprising a compound of Formula (I) and a pharmaceutically acceptable carrier.

In an implementation of the present disclosure, there is provided a compound of Formula (I) for use in treating and/or preventing a neurodegenerative disorder or condition. In a related aspect, the disclosure provides for the use of a compound of Formula (I) for the manufacture of a medicament for treating and/or preventing a neurodegenerative disorder or condition.

In an implementation of the present disclosure, there is provided a pharmaceutical composition comprising a compound of Formula (I) as disclosed herein together with a pharmaceutically acceptable carrier, optionally in combination with one or more other pharmaceutical formulations for use in manufacture of a medicament for inhibiting HDAC enzymes or LSD1 enzymes, or both HDAC enzymes and LSD1 enzymes.

In an implementation of the present disclosure, there is provided a pharmaceutical composition comprising a compound of Formula (I) as disclosed herein together with a pharmaceutically acceptable carrier, optionally in combination with one or more other pharmaceutical formulations for use in manufacture of a medicament for modulating MYC.

In an implementation of the present disclosure, there is provided a method of treating, preventing, modulating or ameliorating a disease or condition mediated by MYC or coREST or LSD1 or HDAC or both LSD1 and HDAC in a subject in need thereof, the method comprising: administering to the subject a therapeutically effective amount of a compound of Formula (I) or the pharmaceutical composition as disclosed herein.

In an implementation of the present disclosure, there is provided a method of treating, preventing, modulating or ameliorating a disease or condition mediated by LSD1 or HDAC or both LSD1 and HDAC in a subject in need thereof, the method comprising: administering to the subject a therapeutically effective amount of a compound of Formula (I) or the pharmaceutical composition as disclosed herein.

In an implementation of the present disclosure, there is provided a method for treating, preventing, modulating, or ameliorating a disease or condition associated with MYC or coREST in a subject in need thereof, the method comprising: administering to the subject a therapeutically effective amount of a compound of Formula (I) or the pharmaceutical composition as disclosed herein.

In an implementation of the present disclosure, there is provided a method of inhibiting both LSD1 and HDAC in a cell in a subject in need thereof, the method comprising: administering to the subject a therapeutically effective amount of the compound of Formula (I) or the pharmaceutical composition as disclosed herein.

In an implementation of the present disclosure, there is provided a method of inhibiting transcriptional cofactor in a subject in need thereof, the method comprising: administering to the subject a therapeutically effective amount of the compound of Formula (I) or the pharmaceutical composition as disclosed herein.

In an implementation of the present disclosure, there is provided a method of treatment and/or prevention of a disease or a condition mediated by both LSD1 and HDAC or a proliferative disorder or cancer, comprising administering to a subject suffering from the disease or condition mediated by both LSD1 and HDAC or the proliferative disorder or cancer, a therapeutically effective amount of the compound of Formula (I) or the pharmaceutical composition as disclosed herein.

In an implementation of the present disclosure, there is provided a method as disclosed herein, wherein the disease or condition is selected from breast cancer, prostate cancer, pancreatic cancer, gastric cancer, lung cancer, colon cancer, rectal cancer, esophagus cancer, duodenal cancer, tongue cancer, pharyngeal cancer, brain tumor, neurinoma, clear cell carcinoma, non-small cell lung cancer, small cell lung cancer, liver cancer, liver metastasis, kidney cancer, bile duct cancer, uterine body cancer, cervical cancer, ovarian cancer, urinary bladder, skin cancer, hemangioma, malignant lymphoma, malignant melanoma, thyroid cancer, bone tumor, vascular fibroma, glioblastoma, sarcoma, neuroendocrine tumors, retinoblastoma, penile cancer, pediatric solid cancer, renal cell carcinoma, lymphoma, myeloma and leukemia including acute myelogenous leukemia (AML), chronic myelogenous leukemia (CML), chronic neutrophilic leukemia, chronic eosinophilic leukemia, chronic lymphocytic leukemia (CLL), acute lymphoblastic leukemia (ALL), hairy cell leukemia, cutaneous T-cell lymphoma (CTCL), multiple myeloma (MM), Myeloproliferative neoplasms (MPN), polycythemia vera (PV), myeloproliferative dysplastic syndrome, essential thrombocythemia (ET), essential thrombocytosis and myelofibrosis (MF), chronic myelogenous leukemia (CML), chronic neutrophilic leukemia (CNL), chronic eosinophilic leukemia (CEL), cancers with specific mutations in the SWI/SNF complex, ARID1A, ARID1B, SMARCA4, SMARCA2, cancers with mutations in specific oncogenes, EGFR, KRAS, RET, cancers with differential expression of certain genes, LAPTM5, RNASE6, IL-16, APOBEC3 and TYROBP, depression, Alzheimer's disease, Huntington disease, Parkinson's disease, Amyotrophic lateral sclerosis, Dementia with lewy bodies, Frontotemporal dementia or MYC amplified tumor.

In an implementation of the present disclosure, there is provided a method of treating a subject with a tumor, the method comprising: administering to the subject a therapeutically effective amount of a compound of Formula (I) or the pharmaceutical composition as disclosed herein.

In an implementation of the present disclosure, there is provided a method of treating a subject with a tumor as disclosed herein, wherein the tumor is a neuroendocrine tumor and the neuroendocrine tumor is a small cell lung cancer, neuroendocrine prostate cancer or MYC amplified tumor.

In an implementation of the present disclosure, there is provided a method of treating a subject with cancer, the method comprising: administering to the subject a therapeutically effective amount of a compound of Formula (I) or the pharmaceutical composition as disclosed herein.

In an implementation of the present disclosure, there is provided a method of treating a subject with cancer as disclosed herein, wherein the cancer is selected from small cell lung cancer (SCLC), gastric cancer, pancreatic cancer, lung cancer, liver metastasis and breast cancer.

In an implementation of the present disclosure, there is provided a method of treating a subject with cancer as disclosed herein, wherein the cancer has RB1 mutation. In an implementation of the present disclosure, there is provided a method of treating a subject with cancer as disclosed herein, wherein the cancer is breast cancer, and the breast cancer has RB1 mutation.

In an implementation of the present disclosure, there is provided a compound which may be administered in combination therapy by combining the compound of Formula (I) with one or more separate agents, not limited to targets such as DNA methyltransferase, heat shock proteins (e.g. HSP90), kinase, epigenetic and other matrix metalloproteinases.

“Combination therapy” includes the administration of the subject compounds in further combination with other biologically active ingredients (such as, but are not limited to, different antineoplastic agent) and non-drug therapies (such as, but are not limited to, surgery or radiation treatment). The compounds described herein can be used in combination with other pharmaceutically active compounds, preferably, which will enhance the effect of the compounds of the disclosure. The compounds can be administered simultaneously or sequentially to the other drug therapy.

In an implementation of the present disclosure, the subject compounds may be combined with the antineoplastic agents (e.g. small molecules, cytotoxic reagents, non-cytotoxic reagents, monoclonal antibodies, antisense RNA and fusion proteins) that inhibit one or more biological targets. Such combination may enhance therapeutic efficacy over the efficacy achieved by any of the agents alone and may prevent or delay the appearance of resistant variants.

In an implementation of the present disclosure, the subject compounds may be combined with immuno-oncology drugs or “immune checkpoint inhibitors agents” or “immune modulators agents” not restricting to CD27, CD28, CD40, CD 122, CD96, CD73, CD47, OX40, GITR, CSF1R, JAK, PI3K delta, PI3K gamma, TAM arginase, CD137 (also known as 4-1BB), ICOS, A2AR, B7-H3, B7-H4, BTLA, CTLA-4, LAG3, TIM3, VISTA, PD-1, PD-L1 and PD-L2.

Although the subject matter has been described in considerable detail with reference to certain embodiments thereof, other embodiments are possible. As such, the spirit and scope of the disclosure should not be limited to the description of the embodiments contained herein.

### EXAMPLES

The disclosure will now be illustrated with the working examples, which is intended to illustrate the working of disclosure and not intended to take restrictively to imply any limitations on the scope of the present disclosure. Unless defined otherwise, all technical and scientific terms used herein have the same meaning as commonly understood to one ordinary person skilled in the art to which this disclosure belongs. Although methods and materials similar or equivalent to those disclosed herein can be used in the practice of the disclosed methods and compositions, the exemplary methods, devices and materials are disclosed herein. It is to be understood that this disclosure is not limited to particular methods, and experimental conditions described, as such methods and conditions may apply.

### General Modes of Preparation

The following examples provide the details about the synthesis, activities and applications of the compounds of the present disclosure. It should be understood the following is representative only, and that the disclosure is not limited by the details set forth in these examples.

There is also provided a process as shown in the following scheme-1, for the preparation of compounds of the Formula (I), wherein all the groups are as defined earlier. The production method is covered by WO2017/195216, which is hereby incorporated by reference.

The process comprising the steps of:
Step 1: reacting a compound 1 with an aldehyde or ketone in a protic solvent to give an intermediate imine followed by reaction with a reducing agent to give an intermediate compound 2 or alternatively reacting compound 1 with alkyl halide in the presence/absence of base to give intermediate compound 2;
Step 2: hydrolyzing the intermediate compound 2 with an inorganic base to result in the corresponding acid, followed by coupling the acid with activating agents such as EDCI. HCl (1 (3-dimethylaminopropyl)-3-ethylcarbodiimide hydrochloride) and HOBt (1-hydroxybenzotriazole) or (1-propylphosphonic anhydride) T3P/triethylamine and in the presence of the respective amine NH₂R₂ to yield the compound of Formula (I) or alternatively reacting the intermediate compound 2 with NH₂R₂ and an inorganic base to yield the compound of Formula (I)

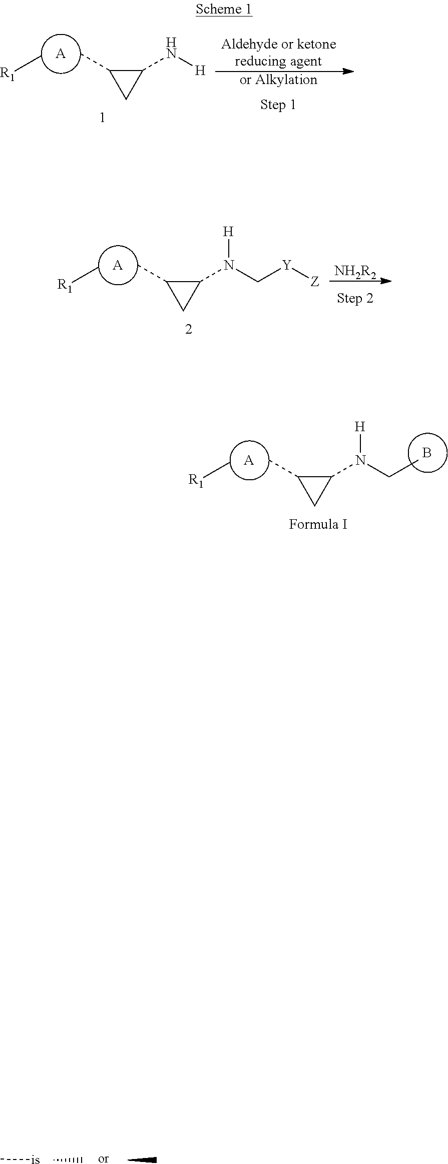

### Synthesis of Intermediates-Amines

### C-1 and C-2: (1R,2S)-2-(4-fluorophenyl)cyclopropanamine hydrochloride and (1S,2R)-2-(4-fluorophenyl)cyclopropanamine-hydrochloride

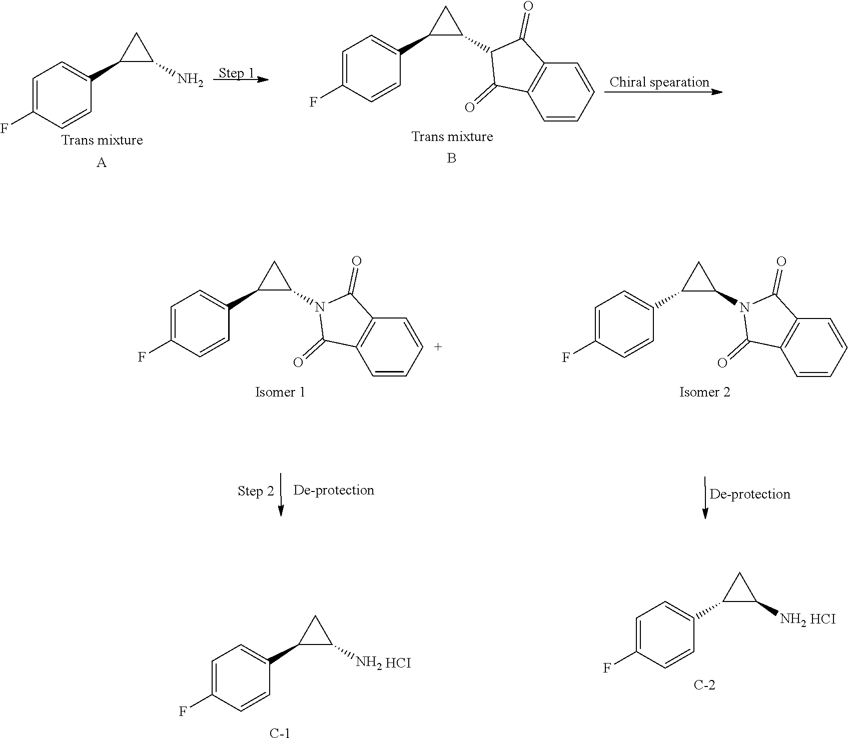

### Step 1: 2-((1S,2R)-2-(4-fluorophenyl)cyclopropyl)isoindoline-1,3-dione and 2-((1R,2S)-2-(4-fluorophenyl)cyclopropyl)isoindoline-1,3-dione-B

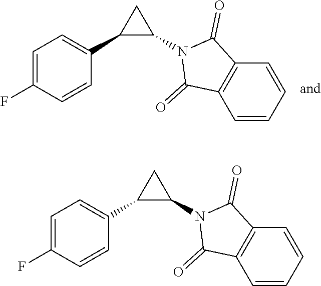 [SMILES: O=C1c2ccccc2C(=O)N1[C@H]1C[C@@H]1c1ccc(F)cc1 ; O=C1c2ccccc2C(=O)N1[C@@H]1C[C@H]1c1ccc(F)cc1]

A mixture of 2-(4-fluoro-phenyl)-cyclopropylamine hydrochloride (A, 1.5 g, 7.99 mmol), isobenzofuran-1,3-dione (1.77 g, 11.99 mmol) and diisopropylethylamine (4.27 mL, 23.97 mmol) was heated in a sealed tube at 150° C. for 12 h and cooled to room temperature. The reaction mixture was diluted with ethylacetate (3×50 mL). The organic portion was washed with water and brine dried over sodium sulphate and concentrated under reduced pressure to afford the crude compound which was purified by column chromatography to afford the racemic product (B, 1.9 g). The racemic product was separated by chiral Prep. HPLC, Chiralpak IA (250 mm×4.6 mm×5 m) using 0.1% TFA in ACN:MeOH (20:80%) solvent to get isomer 1 (0.73 g) and isomer 2 (0.77 g). LC-MS m/z calcd for C₁₇H₁₂FNO₂, 281.1; found 282.2 [M+H]⁺.

### Step 2: (1R,2S)-2-(4-fluorophenyl)cyclopropanamine

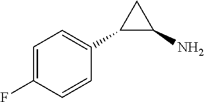 [SMILES: N[C@@H]1C[C@H]1c1ccc(F)cc1]

To a stirred solution of 2-[2-(4-fluoro-phenyl)-cyclopropyl]-isoindole-1,3-dione (isomer 2, 0.77 g, 2.73 mmol) in dichloromethane and ethanol mixture (12 mL, 5:1) was added hydrazine hydrate (0.41 mL, 8.21 mmol) at room temperature and the resulting mixture was stirred at room temperature for 3 h. The progress of the reaction was monitored by TLC. A precipitate formed which was filtered and washed with dichloromethane. The filtrate was evaporated to give the product as yellow oil (0.47 g, 95%). The crude was carried to next step without further purification. LC-MS m/z calcd for C₉H₁₀FN, 151.1; found 152.2 [M+H]⁺.

### C-2 (Isomer 2): (1R,2S)-2-(4-fluorophenyl)cyclopropanamine hydrochloride

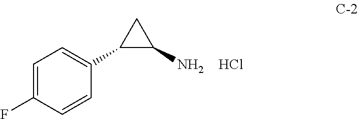 [SMILES: N[C@@H]1C[C@H]1c1ccc(F)cc1 ; Cl]

To a stirred solution of 2-(4-fluoro-phenyl)-cyclopropylamine (chirally pure, 0.47 g, 2.108 mmol) in dioxane (5 mL) was added HCl in dioxane solution (2 mL) at 0° C. and the resulting mixture was stirred at room temperature for 2 h. Cooled to room temperature and the solvent was evaporated to get the residue which was triturated with diethyl ether to afford the product as an off-white solid (C-2, 0.42 g, 72%). LC-MS m/z calcd for C₉H₁₀FN, 151.1; found 152.2 [M+H]⁺.

### C-1 (Isomer 1): (1S,2R)-2-(4-fluorophenyl)cyclopropanamine hydrochloride

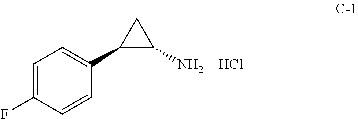 [SMILES: N[C@H]1C[C@@H]1c1ccc(F)cc1 ; Cl]

The compound was synthesized from 2-((1S,2R)-2-(4-fluorophenyl)cyclopropyl)isoindoline-1,3-dione (Isomer 1) by following the same synthesis procedure of (1R,2S)-2-(4-fluorophenyl)cyclopropanamine hydrochloride, LC-MS m/z calcd for C₉H₁₀FN, 151.1; found 152.2 [M+H]⁺.

The present disclosure is further exemplified, but not limited, by the following examples that illustrate the preparation of compounds according to the invention.

### Example 1: Preparation of N-hydroxy-4-((4-((((1R,2S)-2-phenylcyclopropyl)amino)methyl)-1H-pyrazol-1-yl)methyl)benzamide hydrobromide

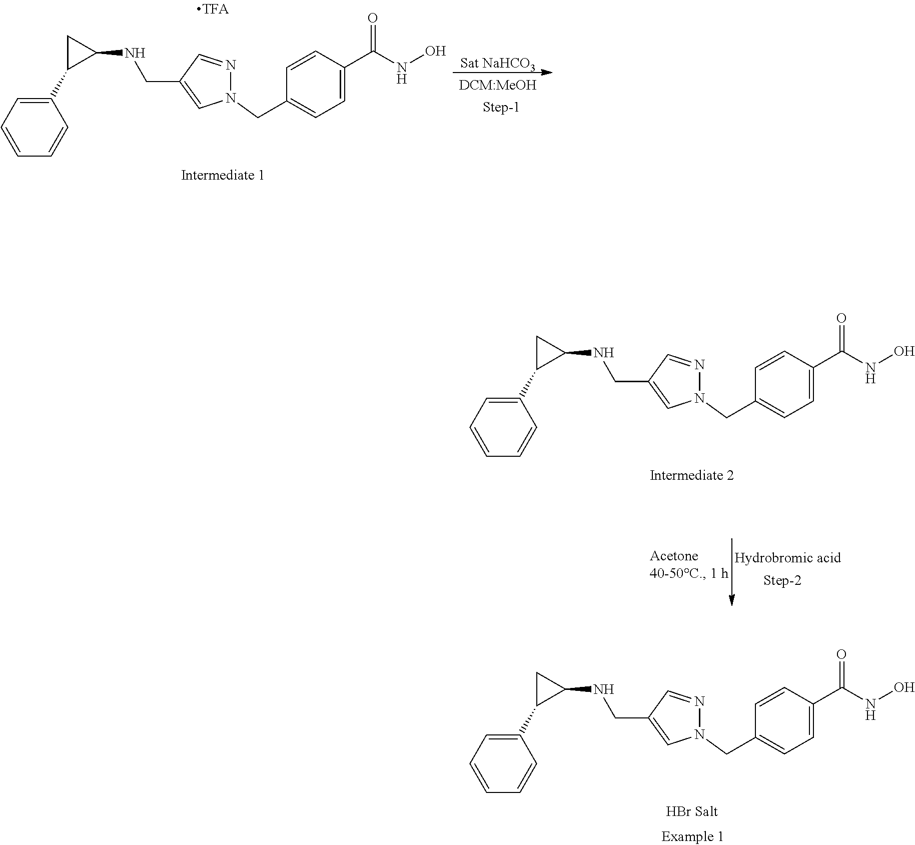

Intermediate 1 was prepared using the chiral pure isomer (1R,2S)-2-phenylcyclopropan-1-amine according to the synthetic methods mentioned in the PCT patent application WO2017/195216.

### Step 1: Synthesis of N-hydroxy-4-((4-((((1R,2S)-2-phenylcyclopropyl)amino)methyl)-1H-pyrazol-1-yl)methyl)benzamide (2)

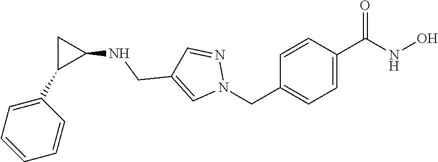 [SMILES: O=C(NO)c1ccc(Cn2cc(CN[C@@H]3C[C@H]3c3ccccc3)cn2)cc1]

To a solution of N-hydroxy-4-((4-((((1R,2S)-2-phenylcyclopropyl)amino)methyl)-1H-pyrazol-1-yl)methyl)benzamide as TFA salt (17.0 g, 37.03 mmol) in 10% methanol-dichloromethane (800 mL) was added aqueous saturated solution of NaHCO₃ (200 mL). Organic layer was separated, dried over anhydrous sodium sulphate and concentrated under vacuum to get the title compound N-hydroxy-4-((4-((((1R,2S)-2-phenylcyclopropyl)amino)methyl)-1H-pyrazol-1-yl)methyl)benzamide (10.5 g, crude). The crude was as such used for next step without further purification.

### Step 2: N-hydroxy-4-((4-((((1R,2S)-2-phenylcyclopropyl)amino)methyl)-1H-pyrazol-1-yl)methyl)benzamide hydrobromide (Example 1)

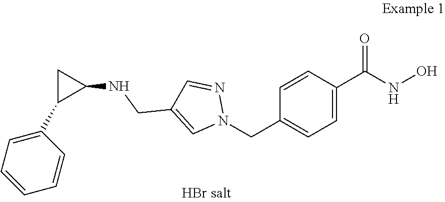 [SMILES: O=C(NO)c1ccc(Cn2cc(CN[C@@H]3C[C@H]3c3ccccc3)cn2)cc1]

To a mixture of N-hydroxy-4-((4-((((1R,2S)-2-phenylcyclopropyl)amino)methyl)-1H-pyrazol-1-yl)methyl)benzamide (2, 1 eq.) in acetone (20 V), hydrobromic acid (1.05 eq, 47% in water) was added and the reaction mixture was stirred at 40° C. for 1 h. The reaction mixture was cooled to 20° C. and the precipitated solid was filtered, washed with acetone (2 V) and dried under vacuum to afford the titled product N-hydroxy-4-((4-((((1R,2S)-2-phenylcyclopropyl)amino)methyl)-1H-pyrazol-1-yl)methyl)benzamide hydrobromide salt (Example 1, 84.33%) as off white solid.

¹HNMR (400 MHz, DMSO-d₆): δ 11.22 (bs, 1H), 9.04 (bs, 2H), 7.93 (s, 1H), 7.71 (d, J=8 Hz, 2H), 7.58 (s, 1H), 7.30-7.18 (m, 5H), 7.12 (d, J=7.6 Hz, 2H), 5.37 (s, 2H), 4.16 (s, 2H), 2.88-2.86 (m, 1H), 2.43-2.38 (m, 1H), 1.47-1.42 (m, 1H), 1.27-1.24 (m, 1H). LC-MS m/z calcd for C₂₂H₂₁N₄O₂, 362.17; found 363.08 [M+H]⁺. HPLC purity 98%.

The following enantiomeric compounds were synthesized as TFA salts using the chiral amines according to the synthetic methods mentioned in the PCT patent application WO2017/195216. The chiral amines were prepared according the synthetic method used for the preparation of intermediate C₁ and C₂ above.

### Example 2: N-hydroxy-4-((4-((((1S,2R)-2-phenylcyclopropyl)amino)methyl)-1H-pyrazol-1-yl)methyl)benzamide

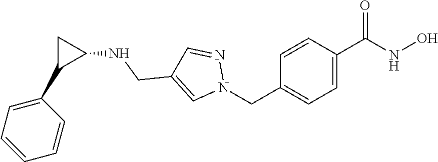

¹HNMR (400 MHz, DMSO-d₆): δ 11.16 (bs, 1H), 9.06 (bs, 3H), 7.87 (s, 1H), 7.70 (d, J=8 Hz, 2H), 7.54 (s, 1H), 7.31-7.19 (m, 5H), 7.12 (d, J=7.6 Hz, 2H), 5.37 (s, 2H), 4.18 (s, 2H), 2.90 (bs, 1H), 2.36 (bs, 1H), 1.42-1.37 (m, 1H), 1.30-1.26 (m, 1H). LC-MS m/z calcd for C₂₁H₂₂N₄O₂, 362.17; found 363.2 [M+H]⁺. HPLC purity 99.8%

### Example 3: N-hydroxy-4-(3-(4-((((1R,2S)-2-(1-phenyl-1H-pyrazol-4-yl)cyclopropyl)amino)methyl)piperidin-1-yl)propyl)benzamide

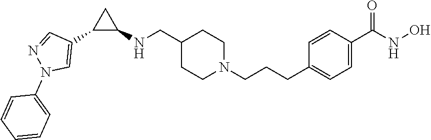 [SMILES: O=C(NO)c1ccc(CCCN2CCC(CN[C@@H]3C[C@H]3c3cnn(-c4ccccc4)c3)CC2)cc1]

¹HNMR (400 MHz, DMSO-d₆): δ 11.14 (bs, 1H), 9.16 (bs, 1H), 8.93 (bs, 2H), 8.36 (s, 1H), 7.75-7.61 (m, 5H), 7.47 (t, J=7.6 Hz, 2H), 7.31-7.24 (m, 3H), 3.03 (bs, 5H), 2.95-2.85 (m, 4H), 2.65 (bs, 2H), 2.31 (bs, 1H), 2.02-1.89 (m, 5H), 1.45-1.34 (m, 3H), 1.25-1.22 (m, 1H). LC-MS m/z calcd for C₂₈H₃₅N₅O₂, 473.3; found 474.5 [M+H]⁺. HPLC purity 98.0%.

### Example 4: N-hydroxy-4-(3-(4-((((1S,2R)-2-(1-phenyl-1H-pyrazol-4-yl)cyclopropyl)amino)methyl)piperidin-1-yl)propyl)benzamide

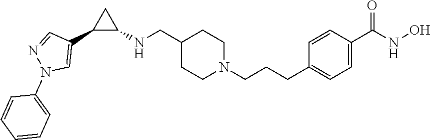 [SMILES: O=C(NO)c1ccc(CCCN2CCC(CN[C@H]3C[C@@H]3c3cnn(-c4ccccc4)c3)CC2)cc1]

¹HNMR (400 MHz, DMSO-d₆): δ 11.14 (bs, 1H), 9.22 (bs, 1H), 8.97 (bs, 2H), 8.36 (s, 1H), 7.75-7.66 (m, 5H), 7.47 (t, J=7.2 Hz, 2H), 7.31-7.26 (m, 3H), 3.54-3.52 (m, 2H), 3.03 (bs, 4H), 2.95-2.85 (m, 3H), 2.65 (bs, 2H), 2.31 (bs, 1H), 2.02-1.89 (m, 5H), 1.45-1.34 (m, 3H), 1.25-1.22 (m, 1H). LC-MS m/z calcd for C₂₈H₃₅N₅O₂, 473.3; found 474.4 [M+H]⁺. HPLC purity 98.39%.

### Example 5: 4-((E)-3-(3-((((1R,2S)-2-(4-fluorophenyl)cyclopropyl)amino)methyl)azetidin-1-yl)-3-oxoprop-1-en-1-yl)-N-hydroxybenzamide

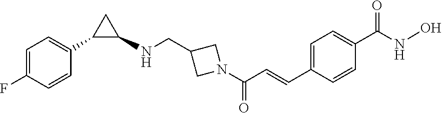 [SMILES: O=C(NO)c1ccc(/C=C/C(=O)N2CC(CN[C@@H]3C[C@H]3c3ccc(F)cc3)C2)cc1]

¹HNMR (400 MHz, DMSO-d₆): δ 11.25 (s, 1H), 9.04 (s, 1H), 8.90 (bs, 2H), 7.77-7.71 (m, 4H), 7.45 (d, J=15.6 Hz, 1H), 7.25-7.21 (m, 2H), 7.13 (t, J=8.8 Hz, 2H), 6.74 (d, J=16 Hz, 1H), 4.47-4.40 (m, 1H), 4.10-4.02 (m, 2H), 3.81-3.76 (m, 1H), 3.45-3.38 (m, 2H), 3.00-2.93 (m, 2H), 1.44-1.38 (m, 1H), 1.33-1.25 (m, 1H). 1H merged with DMSO residual peak. LC-MS m/z calcd for C₂₃H₂₄FN₃O₃, 409.18; found 410.2 [M+H]⁺. HPLC purity 99.56%.

### Example 6: 4-((E)-3-(3-((((1S,2R)-2-(4-fluorophenyl)cyclopropyl)amino)methyl)azetidin-1-yl)-3-oxoprop-1-en-1-yl)-N-hydroxybenzamide

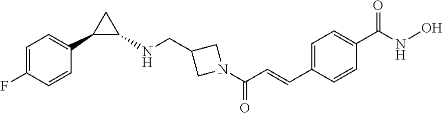 [SMILES: O=C(NO)c1ccc(/C=C/C(=O)N2CC(CN[C@H]3C[C@@H]3c3ccc(F)cc3)C2)cc1]

¹HNMR (400 MHz, DMSO-d₆): δ 11.25 (s, 1H), 9.04 (bs, 1H), 8.90 (bs, 2H), 7.77-7.71 (m, 4H), 7.45 (d, J=15.6 Hz, 1H), 7.25-7.22 (m, 2H), 7.13 (t, J=8.8 Hz, 2H), 6.74 (d, J=16 Hz, 1H), 4.47-4.40 (m, 1H), 4.10-4.02 (m, 2H), 3.80 (bs, 1H), 3.00-2.93 (m, 2H), 1.44-1.38 (m, 1H), 1.33-1.29 (m, 1H). 2H merged with DMSO water peak and 1H merged with DMSO residual peak. LC-MS m/z calcd for C₂₃H₂₄FN₃O₃, 409.18; found 410.1 [M+H]⁺. HPLC purity 98.01%.

### Example 7: N-hydroxy-4-(3-(4-((((1R,2S)-2-(4-(pyrimidin-5-yl)phenyl)cyclopropyl)amino)methyl)piperidin-1-yl)propyl)benzamide

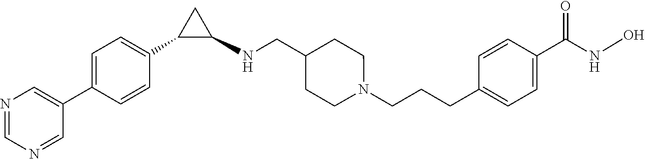 [SMILES: O=C(NO)c1ccc(CCCN2CCC(CN[C@@H]3C[C@H]3c3ccc(-c4cncnc4)cc3)CC2)cc1]

¹HNMR (400 MHz, DMSO-d₆): δ 11.14 (s, 1H), 9.42 (bs, 1H), 9.16 (bs, 2H), 9.11 (bs, 3H), 7. 75 (d, J=8.4 Hz, 2H), 7.69 (d, J=8.0 Hz, 2H), 7.34 (d, J=8.4 Hz, 2H), 7.29 (d, J=8 Hz, 2H), 3.52 (d, J=11.2 Hz, 2H), 3.30-3.18 (m, 1H), 3.10-2.98 (m, 5H), 2.95-2.80 (m, 2H), 2.70-2.62 (m, 2H), 2.55 (bs, 2H), 2.05-1.80 (m, 4H), 1.58-1.50 (m, 1H), 1.46-1.32 (m, 2H). LC-MS m/z calcd for C₂₉H₃₅N₅O₂, 485.27; found 486.4 [M+H]⁺. HPLC purity 99.68%.

### Example 8: N-hydroxy-4-(3-(4-((((1S,2R)-2-(4-(pyrimidin-5-yl)phenyl)cyclopropyl)amino)methyl)piperidin-1-yl)propyl)benzamide

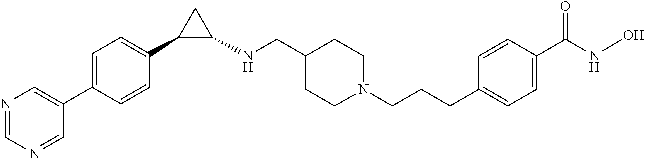 [SMILES: O=C(NO)c1ccc(CCCN2CCC(CN[C@H]3C[C@@H]3c3ccc(-c4cncnc4)cc3)CC2)cc1]

¹HNMR (400 MHz, DMSO-d₆): δ 11.14 (s, 1H), 9.26 (bs, 1H), 9.16 (s, 1H), 9.12 (bs, 2H), 9.00 (bs, 3H), 7.76 (d, J=8 Hz, 2H), 7.70 (d, J=8.0 Hz, 2H), 7.34 (d, J=8 Hz, 2H), 7.30 (d, J=8 Hz, 2H), 3.53 (d, J=11.6 Hz, 2H), 3.30-3.18 (m, 1H), 3.10-2.98 (m, 5H), 2.95-2.80 (m, 2H), 2.70-2.62 (m, 2H), 2.05-1.80 (m, 4H), 1.58-1.50 (m, 1H), 1.46-1.32 (m, 2H). 2H merged with DMSO residual peak. LC-MS m/z calcd for C₂₉H₃₅N₅O₂, 485.27; found 486.4 [M+H]⁺. HPLC purity 99.65%.

### Example 9: N-hydroxy-4-(3-(4-((((1R,2S)-2-(1,3,3-trimethyl-2-oxoindolin-5-yl)cyclopropyl)amino)methyl)piperidin-1-yl)propyl)benzamide

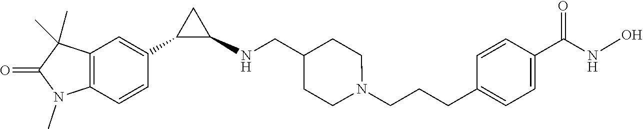 [SMILES: CN1C(=O)C(C)(C)c2cc([C@@H]3C[C@H]3NCC3CCN(CCCc4ccc(C(=O)NO)cc4)CC3)ccc21]

¹HNMR (400 MHz, DMSO-d₆): δ 11.13 (bs, 1H), 9.09 (bs, 1H), 8.84 (bs, 2H), 7.69 (d, J=8 Hz, 2H), 7.29 (d, J=8 Hz, 2H), 7.16 (s, 1H), 7.06 (d, J=7.6 Hz, 1H), 6.92 (d, J=7.6 Hz, 1H), 3.09 (s, 3H), 3.07-2.98 (m, 4H), 2.95-2.82 (m, 4H), 2.68-2.63 (m, 2H), 2.46 (bs, 2H), 1.98-1.91 (m, 4H), 1.90-1.65 (m, 2H), 1.46-1.30 (m. 3H), 1.23 (m. 6H). LC-MS m/z calcd for C₃₀H₄₀N₄O₃, 504.3; found 505.3 [M+H]⁺. HPLC purity 99.32%.

### Example 10: N-hydroxy-4-(3-(4-((((1S,2R)-2-(1,3,3-trimethyl-2-oxoindolin-5-yl)cyclopropyl)amino)methyl)piperidin-1-yl)propyl)benzamide

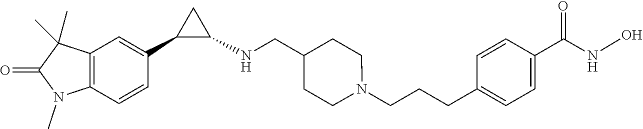 [SMILES: CN1C(=O)C(C)(C)c2cc([C@H]3C[C@@H]3NCC3CCN(CCCc4ccc(C(=O)NO)cc4)CC3)ccc21]

¹HNMR (400 MHz, DMSO-d₆): δ 11.13 (bs, 1H), 9.16 (bs, 1H), 8.88 (bs, 2H), 7.69 (d, J=8 Hz, 2H), 7.29 (d, J=7.6 Hz, 2H), 7.16 (s, 1H), 7.06 (d, J=8 Hz, 1H), 6.91 (d, J=8.4 Hz, 1H), 3.53 (d, J=11.6 Hz, 2H), 3.09 (s, 3H), 3.07-2.98 (m, 4H), 2.95-2.82 (m, 4H), 2.68-2.63 (m, 2H), 2.46 (bs, 2H), 1.98-1.91 (m, 4H), 1.46-1.30 (m, 3H), 1.23 (m, 6H). LC-MS m/z calcd for C₃₀H₄₀N₄O₃, 504.3; found 505.3 [M+H]⁺. HPLC purity 99.86%.

### Example 11: N-hydroxy-4-((4-((((1R,2S)-2-phenylcyclopropyl)amino)methyl)piperidin-1-yl)methyl)benzamide

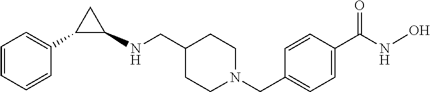 [SMILES: O=C(NO)c1ccc(CN2CCC(CN[C@@H]3C[C@H]3c3ccccc3)CC2)cc1]

¹HNMR (400 MHz, DMSO-d₆): δ 11.27 (bs, 1H), 9.72 (bs, 1H), 9.04 (bs, 2H), 7.81 (d, J=8 Hz, 2H), 7.54 (d, J=8 Hz, 2H), 7.34-7.25 (m, 2H), 7.22-7.14 (m, 3H), 4.30 (s, 2H), 3.39-3.36 (m, 2H), 3.25-2.85 (m, 5H), 2.00-1.80 (m, 4H), 1.50-1.23 (m, 4H). LC-MS m/z calcd for C₂₃H₂₉N₃O₂, 379.2; found 380.5 [M+H]⁺. HPLC purity 99.56%.

### Example 12: N-hydroxy-4-((4-((((1S,2R)-2-phenylcyclopropyl)amino)methyl)piperidin-1-yl)methyl)benzamide

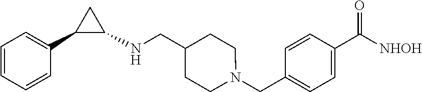

¹HNMR (400 MHz, DMSO-d₆): δ 11.27 (bs, 1H), 9.81 (bs, 1H), 8.99 (bs, 2H), 7.81 (d, J=8 Hz, 2H), 7.54 (d, J=8 Hz, 2H), 7.28 (t, J=7.2 Hz, 2H), 7.22-7.14 (m, 3H), 4.30 (s, 2H), 3.39-3.36 (m, 2H), 3.25-2.85 (m, 5H), 2.00-1.80 (m, 4H), 1.50-1.23 (m, 4H). LC-MS m/z calcd for C₂₃H₂₉N₃O₂, 379.2; found 380.2 [M+H]⁺. HPLC purity 99.67%.

### Example 13: N-hydroxy-4-(3-(4-((((1R,2S)-2-phenylcyclopropyl)amino)methyl)piperidin-1-yl)propyl)benzamide

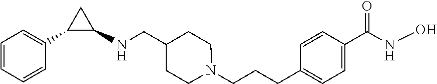 [SMILES: O=C(NO)c1ccc(CCCN2CCC(CN[C@@H]3C[C@H]3c3ccccc3)CC2)cc1]

[0205]¹HNMR (400 MHz, DMSO-d₆): δ 11.09 (bs, 1H), 8.90 (bs, 1H), 7.65 (d, J=8 Hz, 2H), 7.26 (d, J=8 Hz, 2H), 7.20 (t, J=7.6 Hz, 2H), 7.09 (t, J=7.2 Hz, 1H), 7.01 (d, J=6.8 Hz, 2H), 3.97 (s, 3H), 3.09 (bs, 2H), 2.68-2.50 (m, 4H), 2.38-2.30 (m, 2H), 1.88-70 (m, 5H), 1.48 (bs, 1H), 1.28-1.10 (m, 2H), 0.98-0.92 (m, 2H). LC-MS m/z calcd for C₂₅H₃₃N₃O₂, 407.2; found 408.2 [M+H]⁺. HPLC purity 98.30%.

### Example 14: N-hydroxy-4-(3-(4-((((1S,2R)-2-phenylcyclopropyl)amino)methyl)piperidin-1-yl)propyl)benzamide

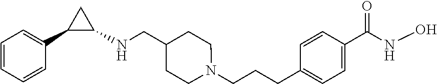 [SMILES: O=C(NO)c1ccc(CCCN2CCC(CN[C@H]3C[C@@H]3c3ccccc3)CC2)cc1]

¹HNMR (400 MHz, DMSO-d₆): δ 11.13 (bs, 1H), 9.30 (bs, 1H), 8.99 (bs, 2H), 7.68 (d, J=8 Hz, 2H), 7.32-7.23 (m, 4H), 7.21 (d, J=7.2 Hz, 1H), 7.18-7.14 (m, 2H), 3.52 (d, J=12 Hz, 2H), 3.28-3.08 (m, 2H), 3.0-2.8 (m, 6H), 2.65 (bs, 2H), 2.0-1.8 (m, 4H), 1.85-1.62 (m, 1H), 1.48-1.22 (m, 4H). LC-MS m/z calcd for C₂₅H₃₃N₃O₂, 407.2; found 408.2 [M+H]⁺. HPLC purity 99.72%.

The following enantiomeric compounds were synthesized as TFA salts using the chiral amines according to the synthetic method mentioned in the PCT patent application WO2017/195216. The chiral amines were prepared according the synthetic method used for the preparation of intermediates C1 and C2 above.

### Example 15: N-(2-aminophenyl)-4-(3-(4-((((1R,2S)-2-(4-fluorophenyl)cyclopropyl)amino)methyl)piperidin-1-yl)propyl)benzamide

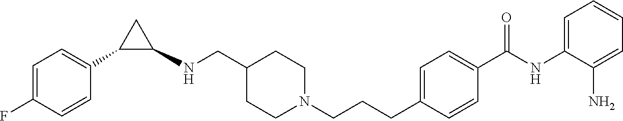 [SMILES: Nc1ccccc1NC(=O)c1ccc(CCCN2CCC(CN[C@@H]3C[C@H]3c3ccc(F)cc3)CC2)cc1]

¹HNMR (400 MHz, DMSO-d₆): δ 9.86 (bs, 1H), 9.49 (bs, 1H), 9.08 (bs, 2H), 7.95 (d, J=7.6 Hz, 2H), 7.37 (d, J=7.6 Hz, 2H), 7.25-7.20 (m, 3H), 7.14-7.07 (m, 3H), 6.98 (d, J=7.6 Hz, 1H), 6.89-6.81 (m, 1H), 3.54 (d, J=11.6 Hz, 2H), 3.30-3.21 (m, 1H), 3.03 (bs, 4H), 2.93-2.88 (m, 3H), 2.71 (t, J=6.8 Hz, 2H), 2.06-1.80 (m, 5H), 1.46-1.35 (m, 3H), 1.30-1.25 (m, 1H). LC-MS m/z calcd for C₃1H₃₇FN₄O, 500.6; found 499.4 [M−H]⁺. HPLC purity 99.67%.

### Example 16: N-(2-aminophenyl)-4-(3-(4-((((1S,2R)-2-(4-fluorophenyl)cyclopropyl)amino)methyl)piperidin-1-yl)propyl)benzamide

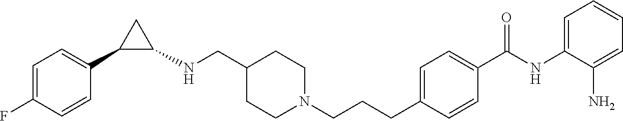 [SMILES: Nc1ccccc1NC(=O)c1ccc(CCCN2CCC(CN[C@H]3C[C@@H]3c3ccc(F)cc3)CC2)cc1]

¹HNMR (400 MHz, DMSO-d₆): δ 9.63 (bs, 1H), 9.05 (bs, 1H), 8.83 (bs, 2H), 7.93 (bs, 2H), 7.37 (bs, 2H), 7.22 (bs, 2H), 7.13 (bs, 3H), 6.98 (bs, 1H), 6.80 (bs, 1H), 6.62 (bs, 1H), 3.56-3.48 (m, 2H), 3.02 (bs, 4H), 2.93 (bs, 4H), 2.65 (bs, 2H), 2.05-1.87 (m, 5H), 1.43-1.39 (m, 3H), 1.30-1.29 (m, 1H). LC-MS m/z calcd for C₃1H₃₇FN₄O, 500.6; found 501.3 [M+H]⁺. HPLC purity 99.76%.

### Example 17: N-(2-aminophenyl)-4-(3-(4-((((1R,2S)-2-(1-isopropyl-1H-pyrazol-4-yl)cyclopropyl)amino)methyl)piperidin-1-yl)propyl)benzamide

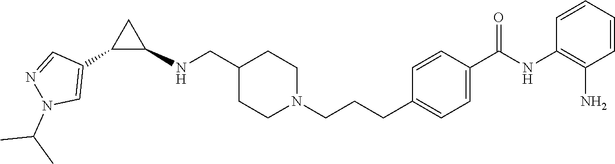 [SMILES: CC(C)n1cc([C@@H]2C[C@H]2NCC2CCN(CCCc3ccc(C(=O)Nc4ccccc4N)cc3)CC2)cn1]

¹HNMR (400 MHz, DMSO-d₆): δ 9.65 (bs, 1H), 9.12 (bs, 1H), 8.77 (bs, 2H), 7.94 (d, J=7.6 Hz, 2H), 7.61 (s, 1H), 7.37 (d, J=6.8 Hz, 2H), 7.30 (s, 1H), 7.16 (d, J=7.2 Hz, 1H), 6.99 (t, J=7.6 Hz, 1H), 6.82 (d, J=7.6 Hz, 1H), 6.73 (t, J=7.2 Hz, 1H), 4.40-4.37 (m, 1H), 3.55-3.52 (m, 2H), 3.08-2.98 (m, 4H), 2.96-2.87 (m, 2H), 2.83-2.78 (m, 1H), 2.75-2.65 (m, 2H), 2.23-2.19 (m, 1H), 2.06-1.90 (m, 5H), 1.46-1.40 (m, 9H), 1.21-1.06 (m, 1H). LC-MS m/z calcd for C₃₁H₄₂N₆O, 514.3; found 515.6 [M+H]⁺. HPLC purity 98.58%.

### Example 18: N-(2-aminophenyl)-4-(3-(4-((((1S,2R)-2-(1-isopropyl-1H-pyrazol-4-yl)cyclopropyl)amino)methyl)piperidin-1-yl)propyl)benzamide

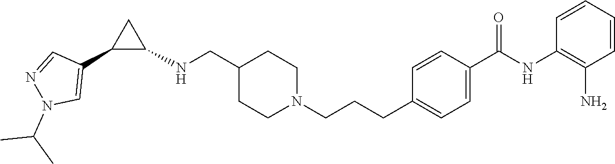 [SMILES: CC(C)n1cc([C@H]2C[C@@H]2NCC2CCN(CCCc3ccc(C(=O)Nc4ccccc4N)cc3)CC2)cn1]

¹HNMR (400 MHz, DMSO-d₆): δ 9.68 (bs, 1H), 9.47 (bs, 1H), 8.96 (bs, 2H), 7.94 (d, J=8 Hz, 2H), 7.61 (s, 1H), 7.37 (d, J=8.4 Hz, 2H), 7.29 (s, 1H), 7.17 (d, J=5.2 Hz, 1H), 7.00 (t, J=7.2 Hz, 1H), 6.83 (d, J=7.6 Hz, 1H), 6.66 (t, J=8 Hz, 1H), 4.43-4.35 (m, 1H), 3.55-3.52 (m, 2H), 3.08-2.98 (m, 4H), 2.96-2.85 (m, 2H), 2.83-2.78 (m, 1H), 2.75-2.65 (m, 2H), 2.28-2.20 (m, 1H), 2.06-1.90 (m, 4H), 1.50-1.40 (m, 3H), 1.34 (d, J=6.4 Hz, 6H), 1.21-1.06 (m, 1H) 0.95-0.9 (m, 1H). LC-MS m/z calcd for C₃₁H₄₂N₆O, 514.3; found 515.2 [M+H]⁺. HPLC purity 98.06%.

### Example 19: N-(2-aminophenyl)-4-((4-((((1R,2S)-2-(4-(1-methyl-6-oxo-1,6-dihydropyridin-3-yl)phenyl)cyclopropyl)amino)methyl)piperidin-1-yl)methyl)benzamide

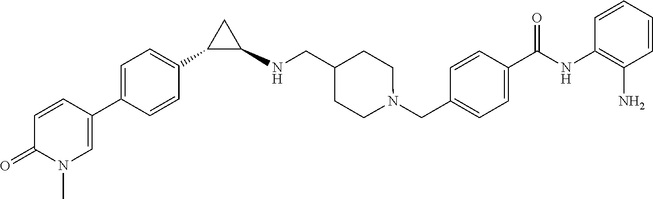 [SMILES: Cn1cc(-c2ccc([C@@H]3C[C@H]3NCC3CCN(Cc4ccc(C(=O)Nc5ccccc5N)cc4)CC3)cc2)ccc1=O]

¹HNMR (400 MHz, DMSO-d₆): δ 9.76 (bs, 1H), 9.60 (bs, 1H), 8.94 (bs, 2H), 8.12-8.04 (m, 3H), 7.80-7.75 (m, 1H), 7.61 (d, J=7.6 Hz, 2H), 7.50 (d, J=8.4 Hz, 2H), 7.22-7.15 (m, 3H), 7.04-6.98 (m, 1H), 6.86-6.78 (m, 1H), 6.64 (bs, 1H), 6.46 (d, J=10 Hz, 1H), 4.37 (s, 2H), 3.49 (s, 3H), 3.42-3.39 (m, 2H), 3.28-3.10 (m, 1H), 3.05-2.92 (m, 5H), 2.02-1.82 (m, 3H), 1.51-1.29 (m, 4H). LC-MS m/z calcd for C₃₅H₃₉N₅O₂, 561.3; found 562.6 [M+H]⁺. HPLC purity 98.08%.

### Example 20: N-(2-aminophenyl)-4-((4-((((1S,2R)-2-(4-(1-methyl-6-oxo-1,6-dihydropvridin-3-yl)phenyl)cyclopropyl)amino)methyl)piperidin-1-yl)methyl)benzamide

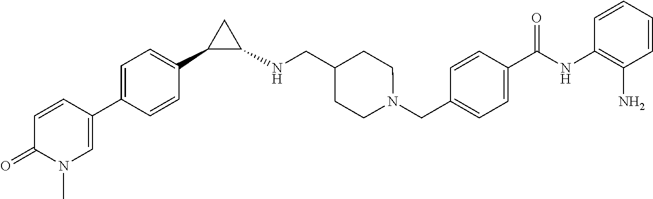 [SMILES: Cn1cc(-c2ccc([C@H]3C[C@@H]3NCC3CCN(Cc4ccc(C(=O)Nc5ccccc5N)cc4)CC3)cc2)ccc1=O]

¹HNMR (400 MHz, DMSO-d₆): δ 9.79 (bs, 2H), 9.05 (bs, 2H), 8.12-8.02 (m, 3H), 7.78 (d, J=9.6 Hz, 1H), 7.61 (d, J=8 Hz, 2H), 7.49 (d, J=8 Hz, 2H), 7.22-7.15 (m, 3H), 7.00 (t, J=7.2 Hz, 1H), 6.83 (d, J=8 Hz, 1H), 6.67 (t, J=7.2 Hz, 1H), 6.46 (d, J=9.2 Hz, 1H), 4.36 (s, 2H), 3.49 (s, 3H), 3.42-3.39 (m, 2H), 3.28-3.10 (m, 1H), 3.05-2.92 (m, 5H), 2.02-1.82 (m, 3H), 1.51-1.29 (m, 4H). LC-MS m/z calcd for C₃₅H₃₉N₅O₂, 561.3; found 559.9 [M−H]⁺. HPLC purity 99.67%.

### Example 21: N-(2-aminophenyl)-4-((4-((((1R,2S)-2-(4-fluorophenyl)cyclopropyl)amino)methyl)-1H-1,2,3-triazol-1-yl)methyl)benzamide

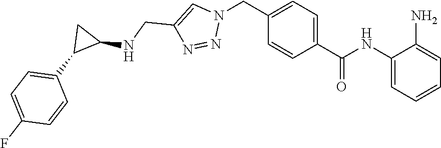 [SMILES: Nc1ccccc1NC(=O)c1ccc(Cn2cc(CN[C@@H]3C[C@H]3c3ccc(F)cc3)nn2)cc1]

¹HNMR (400 MHz, DMSO-d₆): δ 9.73 (bs, 1H), 9.42 (bs, 2H), 8.24 (s, 1H), 7.97 (d, J=8 Hz, 2H), 7.42 (d, J=8.4 Hz, 2H), 7.17-7.09 (m, 5H), 7.01 (t, J=7.6 Hz, 1H), 6.84 (d, J=8 Hz, 1H), 6.68 (bs, 1H), 5.73 (s, 2H), 4.43 (s, 2H), 2.95 (bs, 1H), 2.37 (bs, 1H), 1.40-1.36 (m, 1H), 1.29-1.22 (m, 1H). LC-MS m/z calcd for C₂₆H₂₅FN₆O, 456.2; found 457 [M+H]⁺. HPLC purity 98.96%.

### Example 22: N-(2-aminophenyl)-4-((4-((((1S,2R)-2-(4-fluorophenyl)cyclopropyl)amino)methyl)-1H-1,2,3-triazol-1-yl)methyl)benzamide

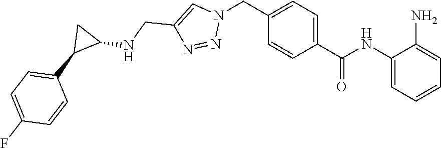 [SMILES: Nc1ccccc1NC(=O)c1ccc(Cn2cc(CN[C@H]3C[C@@H]3c3ccc(F)cc3)nn2)cc1]

¹HNMR (400 MHz, DMSO-d₆): δ 9.81 (bs, 1H), 9.43 (bs, 2H), 8.25 (s, 1H), 7.98 (d, J=8.4 Hz, 2H), 7.43 (d, J=8 Hz, 2H), 7.21-7.05 (m, 6H), 6.93 (bs, 1H), 6.79 (bs, 1H), 5.73 (s, 2H), 4.43 (s, 2H), 2.95 (bs, 1H), 2.37 (bs, 1H), 1.42-1.37 (m, 1H), 1.31-1.20 (m, 1H). LC-MS m/z calcd for C₂₆H₂₅FN₆O, 456.2; found 457.1 [M+H]⁺. HPLC purity 98.92%.

### Example 23: N-(2-aminophenyl)-4-(3-(4-((((1R,2S)-2-(1-phenyl-1H-pyrazol-4-yl)cyclopropyl)amino)methyl)piperidin-1-yl)propyl)benzamide

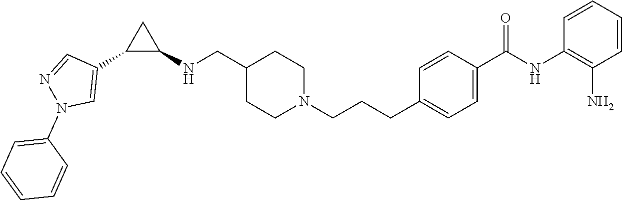 [SMILES: Nc1ccccc1NC(=O)c1ccc(CCCN2CCC(CN[C@@H]3C[C@H]3c3cnn(-c4ccccc4)c3)CC2)cc1]

¹HNMR (400 MHz, DMSO-d₆): δ 9.73 (bs, 1H), 9.28 (bs, 1H), 8.86 (bs, 2H), 8.37 (s, 1H), 7.94 (d, J=8 Hz, 2H), 7.74 (d, J=8 Hz, 2H), 7.65 (s, 1H), 7.47 (t, J=8 Hz, 2H), 7.37 (d, J=8 Hz, 2H), 7.28 (t, J=7.6 Hz, 1H), 7.19 (d, J=6.8 Hz, 1H), 7.06-7.01 (m, 1H), 6.88 (d, J=7.6 Hz, 1H), 6.75-6.68 (m, 1H), 3.58-3.53 (m, 2H), 3.10-3.02 (m, 4H), 2.95-2.85 (m, 3H), 2.71-2.68 (m, 2H), 2.34-2.32 (m, 1H), 2.03-1.90 (m, 5H), 1.45-1.38 (m, 3H), 1.26-1.22 (m, 1H). LC-MS m/z calcd for C₃₄H₄₀N₆O, 548.3; found 549.0 [M+H]⁺. HPLC purity 99.39%.

### Example 24: N-(2-aminophenyl)-4-(3-(4-((((1S,2R)-2-(1-phenyl-1H-pyrazol-4-yl)cyclopropyl)amino)methyl)piperidin-1-yl)propyl)benzamide

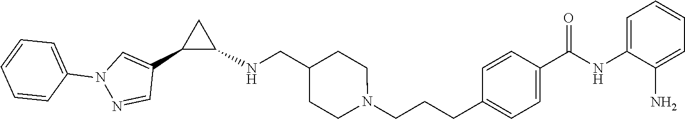 [SMILES: Nc1ccccc1NC(=O)c1ccc(CCCN2CCC(CN[C@H]3C[C@@H]3c3cnn(-c4ccccc4)c3)CC2)cc1]

¹HNMR (400 MHz, DMSO-d₆): δ 9.80 (bs, 1H), 9.41 (bs, 1H), 9.06-8.97 (m, 2H), 8.37 (s, 1H), 7.95 (d, J=8 Hz, 2H), 7.74 (d, J=8 Hz, 2H), 7.66 (s, 1H), 7.47 (t, J=7.6 Hz, 2H), 7.37 (d, J=8.4 Hz, 2H), 7.28 (t, J=7.2 Hz, 1H), 7.22 (d, J=8 Hz, 1H), 7.06 (t, J=7.2 Hz, 1H), 6.93 (d, J=7.6 Hz, 1H), 6.79 (t, J=7.6 Hz, 1H), 3.57-3.53 (m, 2H), 3.28-3.00 (m, 4H), 2.95-2.84 (m, 3H), 2.74-2.68 (m, 2H), 2.38-2.32 (m, 1H), 2.03-1.90 (m, 5H), 1.50-1.32 (m, 3H), 1.25-1.20 (m, 1H). LC-MS m/z calcd for C₃₄H₄₀N₆O, 548.3; found 549.3 [M+H]⁺. HPLC purity 98.63%.

### Example 25: N-(2-aminophenyl)-4-(3-(4-((((1R,2S)-2-(3,4-difluorophenyl)cyclopropyl)amino)methyl)piperidin-1-yl)propyl)benzamide

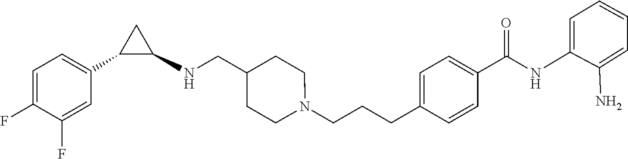 [SMILES: Nc1ccccc1NC(=O)c1ccc(CCCN2CCC(CN[C@@H]3C[C@H]3c3ccc(F)c(F)c3)CC2)cc1]

¹HNMR (400 MHz, DMSO-d₆): δ 9.70 (s, 1H), 9.31 (bs, 1H), 8.99 (bs, 2H), 7.94 (d, J=8 Hz, 2H), 7.37 (d, J=7.6 Hz, 2H), 7.32-7.26 (m, 1H), 7.18 (d, J=7.6 Hz, 1H), 7.07-7.00 (m, 2H), 6.86 (d, J=8 Hz, 1H), 6.70 (t, J=7.6 Hz, 1H), 3.54 (d, J=11.6 Hz, 2H), 3.26-3.18 (bs, 1H), 3.02 (bs, 4H), 2.93-2.87 (m, 2H), 2.71 (t, J=7.6 Hz, 1H), 2.08-1.80 (m, 5H), 1.58-1.30 (m, 4H). 2H merged with DMSO residual peak. LC-MS m/z calcd for C₃₁H₃₆F₂N₄O, 518.2; found 519.5 [M+H]⁺. HPLC purity 99.73%.

### Example 26: N-(2-aminophenyl)-4-(3-(4-((((1S,2R)-2-(3,4-difluorophenyl)cyclopropyl)amino)methyl)piperidin-1-yl)propyl)benzamide

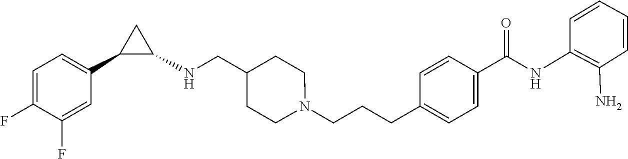 [SMILES: Nc1ccccc1NC(=O)c1ccc(CCCN2CCC(CN[C@H]3C[C@@H]3c3ccc(F)c(F)c3)CC2)cc1]

¹HNMR (400 MHz, DMSO-d₆): δ 9.64 (s, 1H), 9.20 (bs, 1H), 8.97-8.90 (m, 2H), 7.94 (d, J=7.6 Hz, 2H), 7.40-7.26 (m, 4H), 7.15 (d, J=7.6 Hz, 1H), 7.07 (bs, 1H), 6.98 (t, J=7.6 Hz, 1H), 6.81 (d, J=7.6 Hz, 1H), 6.63 (t, J=7.6 Hz, 1H), 3.54 (d, J 11.6 Hz, 2H), 3.26-3.18 (bs, 1H), 3.02 (bs, 4H), 2.93-2.87 (m, 2H), 2.71 (t, J=6.8 Hz, 1H), 2.08-1.80 (m, 5H), 1.52-1.30 (m, 4H). 2H merged with DMSO residual peak. LC-MS m/z calcd for C₃₁H₃₆F₂N₄O, 518.2; found 519.3 [M+H]⁺. HPLC purity 99.80%.

### Example 27: N-(2-aminophenyl)-4-((4-((((1R,2S)-2-(4-(3,5-dimethylisoxazol-4-yl)phenyl)cyclopropyl)amino)methyl)piperidin-1-yl)methyl)benzamide

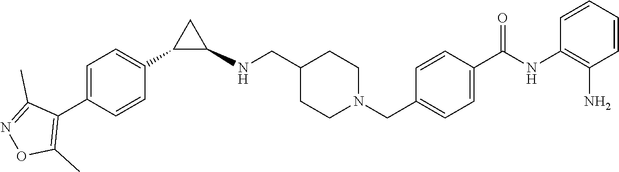 [SMILES: Cc1noc(C)c1-c1ccc([C@@H]2C[C@H]2NCC2CCN(Cc3ccc(C(=O)Nc4ccccc4N)cc3)CC2)cc1]

¹HNMR (400 MHz, DMSO-d₆): δ 9.75 (bs, 1H), 9.53 (bs, 1H), 8.89 (bs, 2H), 8.08 (d, J=7.6 Hz, 2H), 7.61 (d, J=8 Hz, 2H), 7.32-7.26 (m, 4H), 7.16 (d, J=8 Hz, 1H), 7.00 (t, J=7.2 Hz, 1H), 6.82 (bs, 1H), 6.64 (bs, 1H), 4.36 (bs, 2H), 3.40 (bs, 2H), 3.26-3.13 (m, 1H), 3.08-2.91 (m, 5H), 2.36 (s, 3H), 2.18 (s, 3H), 2.00-1.82 (m, 3H), 1.48-1.34 (m, 4H). LC-MS m/z calcd for C₃₅H₃₉N₅O₂, 549.3; found 550.3 [M+H]⁺. HPLC purity 99.86%.

### Example 28: N-(2-aminophenyl)-4-((4-((((1S,2R)-2-(4-(3,5-dimethylisoxazol-4-yl)phenyl)cyclopropyl)amino)methyl)piperidin-1-yl)methyl)benzamide

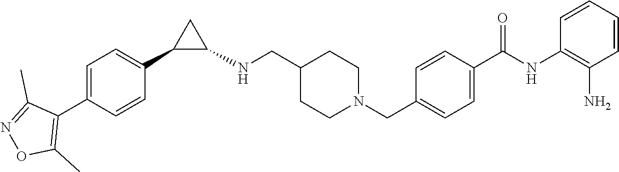 [SMILES: Cc1noc(C)c1-c1ccc([C@H]2C[C@@H]2NCC2CCN(Cc3ccc(C(=O)Nc4ccccc4N)cc3)CC2)cc1]

¹HNMR (400 MHz, DMSO-d₆): δ 9.74 (bs, 1H), 9.55 (bs, 1H), 8.96-8.87 (m, 2H), 8.07 (d, J=7.6 Hz, 2H), 7.61 (d, J=8 Hz, 2H), 7.32-7.26 (m, 4H), 7.15 (d, J=7.2 Hz, 1H), 6.99 (t, J=7.6 Hz, 1H), 6.80 (d, J=8 Hz, 1H), 6.62 (t, J=6.8 Hz, 1H), 4.36 (bs, 2H), 3.43 (bs, 2H), 3.26-3.13 (m, 1H), 3.08-2.90 (m, 5H), 2.36 (s, 3H), 2.18 (s, 3H), 2.00-1.80 (m, 3H), 1.48-1.34 (m, 4H). LC-MS m/z calcd for C₃₅H₃₉N₅O₂, 549.3; found 550.3 [M+H]⁺. HPLC purity 99.39%.

### Example 29: N-(2-aminophenyl)-4-((4-((((1R,2S)-2-phenylcyclopropyl)amino)methyl)-1H-1,2,3-triazol-1-yl)methyl)benzamide

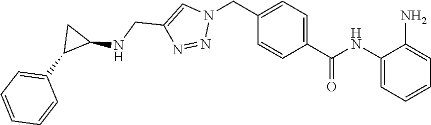 [SMILES: Nc1ccccc1NC(=O)c1ccc(Cn2cc(CN[C@@H]3C[C@H]3c3ccccc3)nn2)cc1]

¹HNMR (400 MHz, DMSO-d₆): δ 9.75 (bs, 1H), 9.43 (bs, 2H), 8.24 (s, 1H), 7.97 (d, J=8 Hz, 2H), 7.42 (d, J=7.6 Hz, 2H), 7.30-7.26 (m, 2H), 7.22-7.18 (m, 2H), 7.11 (d, J=7.2 Hz, 2H), 7.01 (bs, 1H), 6.86 (bs, 1H), 6.71 (bs, 1H), 5.70 (s, 2H), 4.43 (s, 2H), 2.98 (bs, 1H), 2.37 (bs, 1H), 1.41-1.39 (m, 1H), 1.28-1.26 (m, 1H). LC-MS m/z calcd for C₂₆H₂₆N₆O, 438.2; found 439 [M+H]⁺. HPLC purity 99.17%.

### Example 30: N-(2-aminophenyl)-4-((4-((((1R,2S)-2-phenylcyclopropyl)amino)methyl)-1H-pyrazol-1-yl)methyl)benzamide

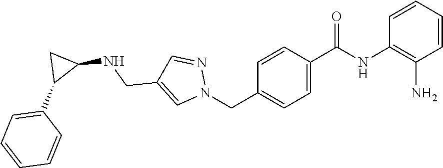 [SMILES: Nc1ccccc1NC(=O)c1ccc(Cn2cc(CN[C@@H]3C[C@H]3c3ccccc3)cn2)cc1]

¹HNMR (400 MHz, DMSO-d₆): δ 9.80 (s, 1H), 9.11-9.06 (m, 2H), 7.95 (d, J=8 Hz, 2H), 7.92 (s, 1H), 7.57 (s, 1H), 7.37 (d, J=11.6 Hz, 2H), 7.29 (d, J=7.6 Hz, 2H), 7.24-7.19 (m, 2H), 7.15-7.13 (m, 2H), 7.07-7.03 (m, 1H), 6.90 (d, J=7.6 Hz, 1H), 6.75 (t, J=7.2 Hz, 1H), 5.42 (s, 2H), 4.21 (t, J=6 Hz, 2H), 2.95-2.88 (m, 1H), 2.40-2.35 (m, 1H), 1.44-1.39 (m, 1H), 1.33-1.29 (m, 1H). LC-MS m/z calcd for C₂₆H₂₆N₆O, 437.5; found 438.3 [M+H]⁺. HPLC purity 92.63%.

### Example 31: TR-FRET Assay for LSD1 (Perkin Elmer)

LSD1 enzyme was produced in house. Tranylcypromine (TCP), LSD1 inhibitor was procured from Selleckchem. LSD1 enzyme, TCP and Biotinylated peptide substrate were diluted in assay buffer just before use. 2× inhibitor (10 μL, diluted in assay buffer) or Assay Buffer, and 5 nM enzyme were added to a 96 well plate and incubated at room temperature for 30 min. 5 μL of biotinylated Histone H3K4me1 peptide (4×) was added to each well and incubated at room temperature (RT) for 1 hour. Stop Solution containing 300 μM tranylcypromine in 1×LANCE Detection Buffer was added to the wells and incubated for 5 min at RT. Then, Detection mixture containing 2 nM Eu-Ab and 50 nM ULight-Streptavidin in 1×LANCE Detection Buffer was prepared and added to the reaction mixture. This mixture was incubated for 1 hour at room temperature. Readings were taken with the Pherastar Reader in TR-FRET mode (excitation at 337 nm & emission at A-665 nm, B-620 nM).

### Example 32: Histone Deacetylase Assay (BPS Biosciences)

Histone deacetylase assay was done as per manufacturer's instructions. Briefly, assay buffer, 200 uM HDAC substrate (fluorogenic HDAC acetylated peptide substrate for class I HDACs (HDACs 1, 2 and 3) and class 2b HDACs (HDACs 6 and 10) and 1% BSA are taken as a master mixture and aliquoted as 40 uL per well. Compounds (10×) were diluted in assay buffer and were added to respective wells of a black 96 well plate. For HDAC1 (1.4 ng/μL) and for HDAC6 (7 ng/μL) human recombinant enzyme was thawed on ice and 5 μL of respective enzyme was added per well. The plate was incubated at 37° C. for 1 hour. Developer solution was then added (50 μL per well) and incubated at room temperature for 10 minutes. Fluorescence was measured at an excitation wavelength of 350-380 nm and emission wavelength of 440-480 nm.

As described above, compounds were tested for LSD1, HDAC1 and HDAC6 enzyme inhibitory activities.

TABLE 1

| | LSD1 | HDAC6 | HDAC1 |
|---|---|---|---|
| Compound No. | IC₅₀ (μM) | IC₅₀ (μM) | IC₅₀ (μM) |
| 1 | 0.055 | 0.012 | 0.923 |
| 2 | 1.342 | 0.178 | 1.55 |
| 3 | 0.009 | 0.088 | ND |
| 4 | 0.005 | 0.072 | ND |
| 5 | 0.031 | 0.084 | ND |
| 6 | 0.147 | 0.144 | ND |
| 7 | 0.006 | 0.151 | 4.53 |
| 8 | 0.008 | 0.159 | 2.47 |
| 9 | 0.403 | 0.192 | ND |
| 10 | 1.21 | 0.135 | ND |
| 11 | 0.004 | 0.031 | ND |
| 12 | 0.003 | 0.054 | ND |
| 13 | 0.004 | 0.031 | ND |
| 14 | 0.006 | 0.069 | ND |
| 15 | 0.009 | ND | 7.72 |
| 16 | 0.005 | ND | 0.295 |
| 17 | 0.101 | ND | 1.04 |
| 18 | 0.139 | ND | 2.89 |
| 19 | 0.006 | ND | ND |
| 20 | 0.020 | ND | ND |
| 21 | 0.069 | ND | ND |
| 22 | 0.854 | ND | ND |
| 23 | 0.010 | ND | 0.262 |
| 24 | 0.006 | ND | 8.60 |
| 25 | 0.004 | ND | ND |
| 26 | 0.005 | >10 | ND |
| 27 | 0.006 | ND | ND |
| 28 | 0.029 | ND | ND |
| 29 | 0.671 | ND | ND |
| 30 | 0.027 | >10 | 0.181 |

Based on the activity data shown in the above table, it is evident that among the pair of enantiomers, one enantiomer showed superior potency compared to the other enantiomer for LSD1 and HDAC inhibition.

### Example 33

### Western Blotting

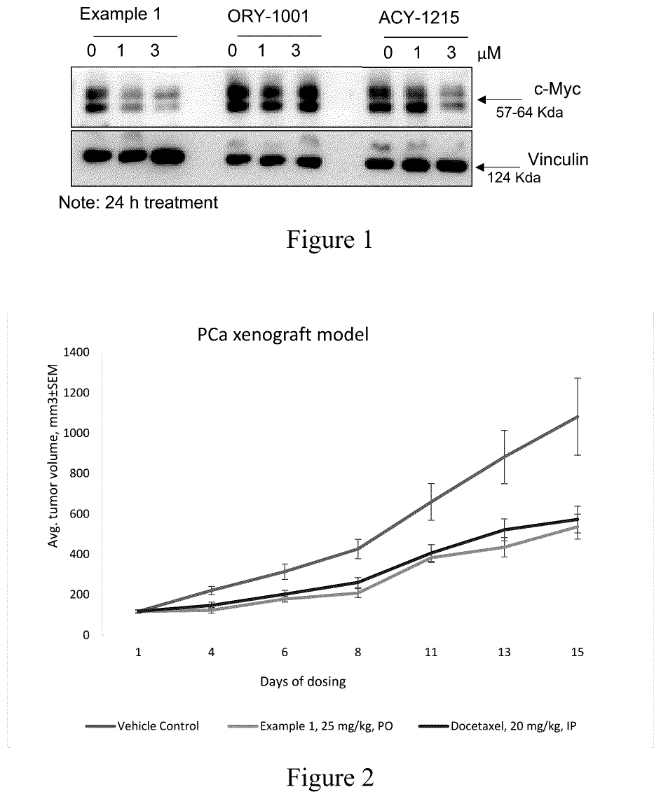

Cells or Tissue samples were homogenized in RIPA buffer (150 mM Tris-HCl, 150 mM NaCl, 1% NP-40, 0.5% sodium deoxycholate, 0.1% SDS, 0.5 mM PMSF, 1× protease inhibitor cocktail) and 5-10 μg of protein was loaded for 10% SDS-PAGE. Proteins were then transferred to a nitrocellulose membrane and then probed with respective antibodies. The bands of interest were visualized by chemiluminescence. Antibodies used were Acetyl α-tubulin, c-Myc, and vinculin (FIG. 1).

### In Vivo Studies

Antitumor efficacy studies of Example 1 were demonstrated in tumor model For prostate cancer (Pca), xenograft models were developed in Male SCID mice (6-7 weeks old, Vivo Biotech, India [under the license from Taconic]) by injecting subcutaneously with 5×10⁶ prostate cancer cells (22RV1) cells in 100 μL of serum-free medium mixed with 1:1 ratio of matrigel (BD, USA). When the tumors reached an approximate volume of 100 mm³, the mice were randomized into different treatment groups (n=8) with a comparable average tumor volume across the groups. Mice injected with 22RV1 were either treated with a dosing vehicle (0.5% methylcellulose) or Example 1 at 25 mg/kg; PO; QD, Docetaxel at 20 mg/kg, IP, QD (FIG. 2).

Dual inhibition of both LSD1/HDAC6 with compounds of Formula (I) as opposed to single target inhibition was able to effectively downregulate MYC level and achieve efficacy in MYC amplified models in vivo and in vitro. This novel mechanism increased the potential population of neuroendocrine patients that could be sensitive to this compound, going beyond the proof of principle already established preclinically and clinically by single target LSD1 inhibitors.

### INCORPORATION BY REFERENCE

All publications and patents mentioned herein are hereby incorporated by reference in their entirety as if each individual publication or patent were specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound of Formula (I): 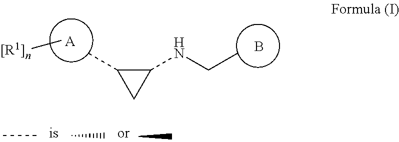
its analogs, tautomeric forms, stereoisomers, enantiomers, diastereomers and pharmaceutically acceptable salts thereof, wherein,
ring A is selected from a group consisting of C₆₋₁₀aryl, C₁₋₆heteroaryl, and C₂₋₁₀ heterocyclyl, wherein, C₁₋₆heteroaryl, and C₂₋₁₀heterocyclyl has one or more heteroatoms selected from N, O or S;
R₁ is independently selected from a group consisting of hydrogen, halogen, hydroxy, nitro, cyano, azido, nitroso, oxo, thioxo, —SO₂—, amino, hydrazino, formyl, C₁₋₈alkyl, C₁₋₈haloalkyl, C₁₋₈alkoxy, C₁₋₈haloalkoxy, C₇₋₁₂arylalkoxy, C₃₋₈cycloalkyl, C₃₋₈cycloalkyloxy, C₆₋₁₀ aryl, C₂₋₁₀heterocyclyl, and C₁₋₆heteroaryl; wherein, C₂₋₁₀heterocyclyl, and C₁₋₆heteroaryl has one or more heteroatoms selected from N, O or S;
wherein, C₇₋₁₂arylalkoxy, C₁₋₈alkyl, C₆₋₁₀aryl, C₁₋₆heteroaryl, C₂₋₁₀heterocyclyl, and C₃₋₈cycloalkyl, is independently optionally substituted with one or more of the groups selected from C₁₋₈ alkyl, C₆₋₁₀aryl, C₁₋₆heteroaryl, C₂₋₁₀heterocyclyl, oxo, C₃₋₈ cycloalkyl, halogen, hydroxy, amino, or cyano;
ring B is 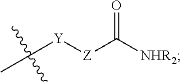
Y is selected from a group consisting of C₁₋₈alkyl, C₂₋₈alkenyl, C₂₋₈alkynyl, C₆₋₁₀ aryl, C₁₋₆heteroaryl, C₂₋₁₀heterocyclyl, C₃₋₈cycloalkyl, —CO, and —CO—C₂₋₁₀heterocyclyl; wherein C₁₋₆heteroaryl, C₂₋₁₀heterocyclyl, and —CO—C₂₋₁₀heterocyclyl has one or more heteroatoms selected from N, O or S;
wherein, C₁₋₈alkyl, C₆₋₁₀aryl, C₁₋₆heteroaryl, C₂₋₁₀heterocyclyl, and C₃₋₈cycloalkyl, is independently optionally substituted with one or more of the groups selected from C₁₋₈ alkyl, oxo, C₃₋₈ cycloalkyl, halogen, hydroxy or cyano;
Z is selected from a group consisting of C₁₋₈alkyl, C₂₋₈alkenyl, C₇₋₁₂-alkylaryl, C₂₋₁₀ heterocyclyl, C₇₋₁₂-arylalkenyl, C₂₋₁₂-alkylheteroaryl, —CO—C₇₋₁₂alkylaryl, —CO—C₇₋₁₂alkenylaryl, —CONR₆—C₁₋₈alkyl, C₆₋₁₀aryl, C₁₋₆heteroaryl, —CO—C₂₋₁₀heterocyclyl, —NR₆—C₆₋₁₀aryl, —NR₆—C₁₋₆heteroaryl, —O—C₆₋₁₀aryl, —O—C₁₋₆heteroaryl, —CONR₆—C₇₋₁₂alkylaryl, —SO₂—C₆₋₁₀aryl, —SO₂—C₇₋₁₂alkylaryl, and —NR₆SO₂—C₇₋₁₂alkylaryl; wherein C₂₋₁₀heterocyclyl, C₂₋₁₂-alkylheteroaryl, C₁₋₆heteroaryl, —CO—C₂₋₁₀heterocyclyl, —NR₆—C₁₋₆heteroaryl, and —O—C₁₋₆heteroaryl has one or more heteroatoms selected from N, O or S;
R₆ is hydrogen or C₁₋₈ alkyl;
R₂ is selected from a group consisting of hydroxy, aniline, C₆₋₁₀aryl, amino C₆₋₁₀ aryl, and amino C₁₋₆heteroaryl; wherein amino C₁₋₆heteroaryl has one or more heteroatoms selected from N, O or S;
wherein, aniline, C₆₋₁₀aryl, amino C₆₋₁₀aryl, and amino C₁₋₆heteroaryl, is independently optionally substituted with one or more of the groups selected from amino, C₁₋₈alkyl, halogen, hydroxy or cyano; and
n is 1 to 4.

2. The compound as claimed in claim 1, having the structure depicted in Formula (IA) 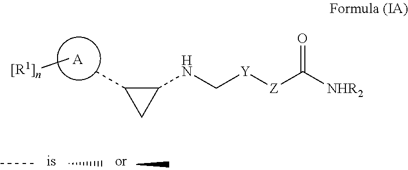
its analogs, tautomeric forms, stereoisomers, enantiomers, diastereomers and pharmaceutically acceptable salts thereof,
wherein,
ring A is selected from a group consisting of C₆₋₁₀aryl, C₁₋₆heteroaryl, and C₂₋₁₀ heterocyclyl, wherein, C₁₋₆heteroaryl, and C₂₋₁₀heterocyclyl has one or more heteroatoms selected from N, O or S;
R₁ is independently selected from a group consisting of hydrogen, halogen, oxo, C₁₋₈alkyl, C₆₋₁₀aryl, C₂₋₁₀heterocyclyl, and C₃₋₆heteroaryl; wherein C₂₋₁₀heterocyclyl and C₃₋₆heteroaryl has one or more heteroatoms selected from N, O or S;
wherein, C₃₋₆heteroaryl, and C₂₋₁₀heterocyclyl, is independently optionally substituted with one or more oxo or C₁₋₈alkyl;
Y is selected from a group consisting of C₁₋₆heteroaryl, C₂₋₁₀heterocyclyl, and —CO; wherein C₁₋₆heteroaryl, and C₂₋₁₀heterocyclyl has one or more heteroatoms selected from N, O or S; wherein, C₁₋₆heteroaryl, and C₂₋₁₀heterocyclyl is independently optionally substituted with one or more oxo or C₁₋₈ alkyl;
Z is selected from a group consisting of C₁₋₈alkyl, C₂₋₈alkenyl, C₇₋₁₂-alkylaryl, C₂₋₁₀ heterocyclyl, —CO—C₇₋₁₂alkylaryl, —CO—C₇₋₁₂alkenylaryl, and C₁₋₆heteroaryl; wherein C₂₋₁₀ heterocyclyl, and C₁₋₆heteroaryl has one or more heteroatoms selected from N, O or S;
R₂ is hydroxy or C₆₋₁₀aryl; wherein, C₆₋₁₀aryl, is substituted with amino; and n is 1 to 4.

3. The compound as claimed in claim 1, wherein, ring A is selected from a group consisting of C₆₋₈ aryl, C₃₋₅heteroaryl, and C₅-9heterocyclyl, wherein, C₃₋₅heteroaryl, and C₅₋₉heterocyclyl has one or more heteroatoms selected from N, O or S;
R₁ is independently selected from a group consisting of hydrogen, halogen, oxo, C₁₋₄alkyl, C₆₋₈aryl, C₅₋₉heterocyclyl, and C₃₋₅heteroaryl; wherein C₅₋₉heterocyclyl and C₃₋₅heteroaryl has one or more heteroatoms selected from N, O or S;
wherein, C₅₋₉heterocyclyl and C₃₋₅heteroaryl is independently optionally substituted with one or more oxo or C₁₋₄alkyl;
Y is selected from a group consisting of C₂₋₅heteroaryl, and C₅₋₉heterocyclyl; wherein C₂₋₅heteroaryl, and C₅₋₉heterocyclyl has one or more heteroatoms selected from N, O or S, wherein, C₂₋₅heteroaryl, and C₅₋₉heterocyclyl is independently optionally substituted with one or more oxo or C₁₋₄ alkyl;
Z is selected from a group consisting of C₁₋₄alkyl, C₇₋₉-alkylaryl, and —CO—C₇₋₁₀alkenylaryl;
R₂ is hydroxy or C₆₋₈aryl; wherein, C₆₋₈aryl is substituted with amino; and n is 1 to 4.

4. The compound as claimed in claim 1, selected from a group consisting of:
1. N-hydroxy-4-((4-((((1R,2S)-2-phenylcyclopropyl)amino)methyl)-1H-pyrazol-1-yl)methyl)benzamide hydrobromide;
2. N-hydroxy-4-((4-((((1S,2R)-2-phenylcyclopropyl)amino)methyl)-1H-pyrazol-1-yl)methyl)benzamide;
3. N-hydroxy-4-(3-(4-((((1R,2S)-2-(1-phenyl-1H-pyrazol-4-yl)cyclopropyl)amino)methyl)piperidin-1-yl)propyl)benzamide;
4. N-hydroxy-4-(3-(4-((((1S,2R)-2-(1-phenyl-1H-pyrazol-4-yl)cyclopropyl)amino)methyl)piperidin-1-yl)propyl)benzamide;
5. 4-((E)-3-(3-((((1R,2S)-2-(4-fluorophenyl)cyclopropyl)amino)methyl) azetidin-1-yl)-3-oxoprop-1-en-1-yl)-N-hydroxybenzamide;
6. 4-((E)-3-(3-((((1S,2R)-2-(4-fluorophenyl)cyclopropyl)amino)methyl) azetidin-1-yl)-3-oxoprop-1-en-1-yl)-N-hydroxybenzamide;
7. N-hydroxy-4-(3-(4-((((1R,2S)-2-(4-(pyrimidin-5-yl)phenyl)cyclo propyl)amino)methyl)piperidin-1-yl)propyl)benzamide;
8. N-hydroxy-4-(3-(4-((((1S,2R)-2-(4-(pyrimidin-5-yl)phenyl)cyclo propyl)amino)methyl)piperidin-1-yl)propyl)benzamide;
9. N-hydroxy-4-(3-(4-((((1R,2S)-2-(1,3,3-trimethyl-2-oxoindolin-5-yl)cyclopropyl)amino)methyl)piperidin-1-yl)propyl)benzamide;
10. N-hydroxy-4-(3-(4-((((1S,2R)-2-(1,3,3-trimethyl-2-oxoindolin-5-yl)cyclopropyl)amino)methyl)piperidin-1-yl)propyl)benzamide;
11. N-hydroxy-4-((4-((((1R,2S)-2-phenylcyclopropyl)amino)methyl) piperidin-1-yl)methyl)benzamide;
12. N-hydroxy-4-((4-((((1S,2R)-2-phenylcyclopropyl)amino)methyl) piperidin-1-yl)methyl)benzamide;
13. N-hydroxy-4-(3-(4-((((1R,2S)-2-phenylcyclopropyl)amino)methyl) piperidin-1-yl)propyl)benzamide;
14. N-hydroxy-4-(3-(4-((((1S,2R)-2-phenylcyclopropyl)amino)methyl) piperidin-1-yl)propyl)benzamide;
15. N-(2-aminophenyl)-4-(3-(4-((((1R,2S)-2-(4-fluorophenyl)cyclopropyl) amino)methyl)piperidin-1-yl)propyl)benzamide;
16. N-(2-aminophenyl)-4-(3-(4-((((1S,2R)-2-(4-fluorophenyl)cyclopropyl) amino)methyl)piperidin-1-yl)propyl)benzamide;
17. N-(2-aminophenyl)-4-(3-(4-((((1R,2S)-2-(1-isopropyl-1H-pyrazol-4-yl)cyclopropyl)amino)methyl)piperidin-1-yl)propyl)benzamide;
18. N-(2-aminophenyl)-4-(3-(4-((((1S,2R)-2-(1-isopropyl-1H-pyrazol-4-yl)cyclopropyl)amino)methyl)piperidin-1-yl)propyl)benzamide;
19. N-(2-aminophenyl)-4-((4-((((1R,2S)-2-(4-(1-methyl-6-oxo-1,6-dihydropyridin-3-yl)phenyl)cyclopropyl)amino)methyl)piperidin-1-yl)methyl)benzamide;
20. N-(2-aminophenyl)-4-((4-((((1S,2R)-2-(4-(1-methyl-6-oxo-1,6-dihydropyridin-3-yl)phenyl)cyclopropyl)amino)methyl)piperidin-1-yl)methyl)benzamide;
21. N-(2-aminophenyl)-4-((4-((((1R,2S)-2-(4-fluorophenyl)cyclopropyl) amino)methyl)-1H-1,2,3-triazol-1-yl)methyl)benzamide;
22. N-(2-aminophenyl)-4-((4-((((1S,2R)-2-(4-fluorophenyl)cyclopropyl) amino)methyl)-1H-1,2,3-triazol-1-yl)methyl)benzamide;
23. N-(2-aminophenyl)-4-(3-(4-((((1R,2S)-2-(1-phenyl-1H-pyrazol-4-yl)cyclopropyl)amino)methyl)piperidin-1-yl)propyl)benzamide;
24. N-(2-aminophenyl)-4-(3-(4-((((1S,2R)-2-(1-phenyl-1H-pyrazol-4-yl)cyclopropyl)amino)methyl)piperidin-1-yl)propyl)benzamide;
25. N-(2-aminophenyl)-4-(3-(4-((((1R,2S)-2-(3,4-difluorophenyl) cyclopropyl)amino)methyl)piperidin-1-yl)propyl)benzamide;
26. N-(2-aminophenyl)-4-(3-(4-((((1S,2R)-2-(3,4-difluorophenyl) cyclopropyl)amino)methyl)piperidin-1-yl)propyl)benzamide;
27. N-(2-aminophenyl)-4-((4-((((1R,2S)-2-(4-(3,5-dimethylisoxazol-4-yl)phenyl)cyclopropyl)amino)methyl)piperidin-1-yl)methyl)benzamide;
28. N-(2-aminophenyl)-4-((4-((((1S,2R)-2-(4-(3,5-dimethylisoxazol-4-yl)phenyl)cyclopropyl)amino)methyl)piperidin-1-yl)methyl)benzamide;
29. N-(2-aminophenyl)-4-((4-((((1R,2S)-2-phenylcyclopropyl)amino)methyl)-1H-1,2,3-triazol-1-yl)methyl)benzamide; and
30. N-(2-aminophenyl)-4-((4-((((1R,2S)-2-phenylcyclopropyl)amino)methyl)-1H-pyrazol-1-yl)methyl)benzamide.

5. A process of preparation of compounds of Formula (I) as claimed in claim 1, the process comprising:
Step 1: reacting a compound 1 with an aldehyde or a ketone in a protic solvent to give an intermediate imine followed by reaction with a reducing agent to give an intermediate compound 2 or alternatively reacting compound 1 with an alkyl halide optionally in presence of a base to give an intermediate compound 2; and
Step 2: hydrolyzing the intermediate compound 2 with an inorganic base to result in the corresponding acid, followed by coupling the acid with the respective amine NH₂R₂ in the presence of an activating agent to yield the compound of Formula (I) or alternatively reacting the intermediate compound 2 with NH₂R₂ and an inorganic base to yield the compound of Formula (I), 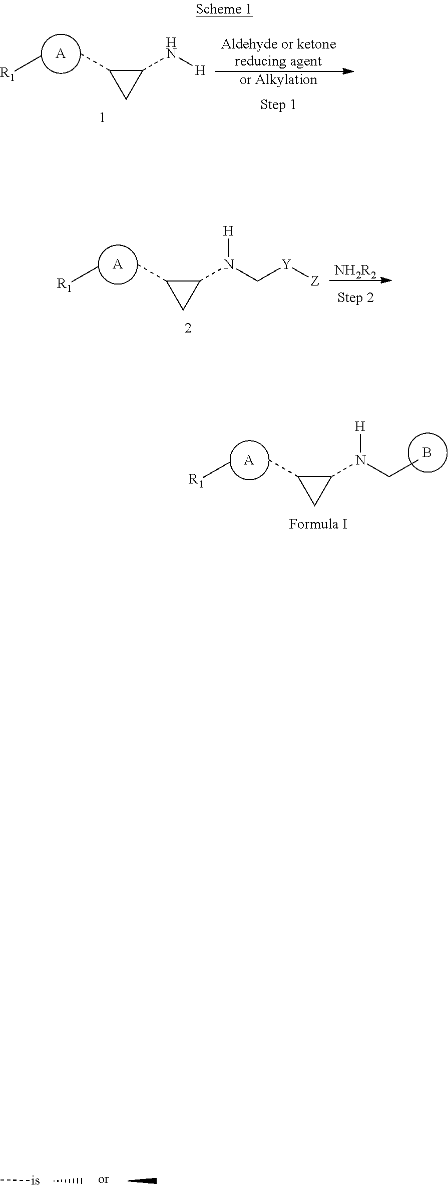
ring A is selected from a group consisting of C₆₋₁₀aryl, C₁₋₆heteroaryl, and C₂₋₁₀ heterocyclyl, wherein, C₁₋₆heteroaryl, and C₂₋₁₀heterocyclyl has one or more heteroatoms selected from N, O or S;
R₁ is independently selected from a group consisting of hydrogen, halogen, hydroxy, nitro, cyano, azido, nitroso, oxo, thioxo, —SO₂—, amino, hydrazino, formyl, C₁₋₈alkyl, C₁₋₈haloalkyl, C₁₋₈alkoxy, C₁₋₈haloalkoxy, C₇₋₁₂arylalkoxy, C₃₋₈cycloalkyl, C₃₋₈cycloalkyloxy, C₆₋₁₀ aryl, C₂₋₁₀heterocyclyl, and C₁₋₆heteroaryl; wherein, C₂₋₁₀heterocyclyl, and C₁₋₆heteroaryl has one or more heteroatoms selected from N, O or S;
wherein, C₇₋₁₂arylalkoxy, C₁₋₈alkyl, C₆₋₁₀aryl, C₁₋₆heteroaryl, C₂₋₁₀heterocyclyl, and C₃₋₈cycloalkyl, is independently optionally substituted with one or more of the groups selected from C₁₋₈ alkyl, C₆₋₁₀aryl, C₁₋₆heteroaryl, C₂₋₁₀heterocyclyl, oxo, C₃₋₈ cycloalkyl, halogen, hydroxy, amino, or cyano;
ring B is 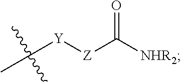
Y is selected from a group consisting of C₁₋₈alkyl, C₂₋₈alkenyl, C₂₋₈alkynyl, C₆₋₁₀ aryl, C₁₋₆heteroaryl, C₂₋₁₀heterocyclyl, C₃₋₈cycloalkyl, —CO, and —CO—C₂₋₁₀heterocyclyl; wherein C₁₋₆heteroaryl, C₂₋₁₀heterocyclyl, and —CO—C₂₋₁₀heterocyclyl has one or more heteroatoms selected from N, O or S;
wherein, C₁₋₈alkyl, C₆₋₁₀aryl, C₁₋₆heteroaryl, C₂₋₁₀heterocyclyl, and C₃₋₈cycloalkyl, is independently optionally substituted with one or more of the groups selected from C₁₋₈ alkyl, oxo, C₃₋₈ cycloalkyl, halogen, hydroxy or cyano;
Z is selected from a group consisting of C₁₋₈alkyl, C₂₋₈alkenyl, C₇₋₁₂-alkylaryl, C₂₋₁₀ heterocyclyl, C₇₋₁₂-arylalkenyl, C₂₋₁₂-alkylheteroaryl, —CO—C₇₋₁₂alkylaryl, —CO—C₇₋₁₂alkenylaryl, —CONR₆—C₁₋₈alkyl, C₆₋₁₀aryl, C₁₋₆heteroaryl, —CO—C₂₋₁₀heterocyclyl, —NR₆—C₆₋₁₀aryl, —NR₆—C₁₋₆heteroaryl, —O—C₆₋₁₀aryl, —O—C₁₋₆heteroaryl, —CONR₆—C₇₋₁₂alkylaryl, —SO₂—C₆₋₁₀aryl, —SO₂—C₇₋₁₂alkylaryl, and —NR₆SO₂—C₇₋₁₂alkylaryl; wherein C₂₋₁₀heterocyclyl, C₂₋₁₂-alkylheteroaryl, C₁₋₆heteroaryl, —CO—C₂₋₁₀heterocyclyl, —NR₆—C₁₋₆heteroaryl, and —O—C₁₋₆heteroaryl has one or more heteroatoms selected from N, O or S;
R₆ is hydrogen or C₁₋₈ alkyl;
R₂ is selected from a group consisting of hydroxy, aniline, C₆₋₁₀aryl, amino C₆₋₁₀ aryl, and amino C₁₋₆heteroaryl; wherein amino C₁₋₆heteroaryl has one or more heteroatoms selected from N, O or S;
wherein, aniline, C₆₋₁₀aryl, amino C₆₋₁₀aryl, and amino C₁₋₆heteroaryl, is independently optionally substituted with one or more of the groups selected from amino, C₁₋₈alkyl, halogen, hydroxy or cyano; and
n is 1 to 4.

6. The process as claimed in claim 5, wherein the activating agent is selected from EDCI. HCl (1(3-dimethylaminopropyl)-3-ethylcarbodiimide hydrochloride), HOBt (1-hydroxybenzotriazole) or (1-propylphosphonic anhydride)T3P/triethylamine; the protic solvent is selected from methanol, ethanol, water, or combinations thereof; and the reducing agent is selected from sodium borohydride, lithium borohydride, aluminum borohydride, or combinations thereof.

7. The process as claimed in claim 5, wherein the base is selected from triethylamine, N, N-diisopropylethylamine, potassium carbonate, pyridine, or combinations thereof; and the inorganic base is selected from potassium carbonate, sodium carbonate, cesium carbonate, or combinations thereof.

8. A pharmaceutical composition comprising the compound as claimed in claim 1 and a pharmaceutically acceptable carrier, optionally in combination with one or more other pharmaceutically acceptable carriers, and wherein the composition is in a form selected from a group consisting of a tablet, capsule, powder, syrup, solution, aerosol and suspension.

9. (canceled)

10. (canceled)

11. A pharmaceutical composition comprising a compound as claimed in claim 1, wherein the compound has enantiomeric excess of higher than 60%, preferably higher than 80%.

12. (canceled)

13. (canceled)

14. A method of treating, preventing, modulating or ameliorating a disease or condition mediated by MYC or coREST or LSD1 or HDAC or both LSD1 and HDAC in a subject in need thereof, the method comprising: administering to the subject a therapeutically effective amount of a compound as claimed in claim 1, optionally with other clinically relevant immune modulator agents or other clinically relevant cytotoxic agents or non-cytotoxic agents.

15. The method as claimed in claim 14, wherein the other clinically relevant immunomodulator agents are selected from a group consisting of CD27, CD28, CD40, CD 122, CD96, CD73, CD47, OX40, GITR, CSF1R, JAK, PI3K delta, PI3K gamma, TAM arginase, CD137 (also known as 4-1BB), ICOS, A2AR, B7-H3, B7-H4, BTLA, CTLA-4, LAG3, TIM3, VISTA, PD-1, PD-L1 and PD-L2.

16. A method of inhibiting both LSD1 and HDAC, or transcriptional cofactor in a cell in a subject in need thereof, the method comprising: administering to the subject a therapeutically effective amount of the compound as claimed in claim 1.

17. A method of treatment and/or prevention of a disease or a condition mediated by both LSD1 and HDAC or a proliferative disorder or cancer, comprising administering to a subject suffering from the disease or condition mediated by both LSD1 and HDAC or the proliferative disorder or cancer, a therapeutically effective amount of the compound as claimed in a claim 1.

18. The method as claimed in claim 14, wherein the disease or condition is selected from breast cancer, prostate cancer, pancreatic cancer, gastric cancer, lung cancer, colon cancer, rectal cancer, esophagus cancer, duodenal cancer, tongue cancer, pharyngeal cancer, brain tumor, neurinoma, clear cell carcinoma, non-small cell lung cancer, small cell lung cancer, liver cancer, liver metastasis, kidney cancer, bile duct cancer, uterine body cancer, cervical cancer, ovarian cancer, urinary bladder, skin cancer, hemangioma, malignant lymphoma, malignant melanoma, thyroid cancer, bone tumor, vascular fibroma, glioblastoma, sarcoma, neuroendocrine tumors, retinoblastoma, penile cancer, pediatric solid cancer, renal cell carcinoma, lymphoma, myeloma and leukemia including acute myelogenous leukemia (AML), chronic myelogenous leukemia (CML), chronic neutrophilic leukemia, chronic eosinophilic leukemia, chronic lymphocytic leukemia (CLL), acute lymphoblastic leukemia (ALL), hairy cell leukemia, cutaneous T-cell lymphoma (CTCL), multiple myeloma (MM), Myeloproliferative neoplasms (MPN), polycythemia vera (PV), myeloproliferative dysplastic syndrome, essential thrombocythemia (ET), essential thrombocytosis and myelofibrosis (MF), chronic myelogenous leukemia (CML), chronic neutrophilic leukemia (CNL), chronic eosinophilic leukemia (CEL), cancers with specific mutations in the SWI/SNF complex, ARID1A, ARID1B, SMARCA4, SMARCA2, cancers with mutations in specific oncogenes, EGFR, KRAS, RET, cancers with differential expression of certain genes, LAPTM5, RNASE6, IL-16, APOBEC3 and TYROBP, depression, Alzheimer's disease, Huntington disease, Parkinson's disease, Amyotrophic lateral sclerosis, Dementia with lewy bodies, Frontotemporal dementia or MYC amplified tumor.

19. The method as claimed in claim 18, wherein the disease or condition is selected from multiple myeloma (MM), myeloproliferative neoplasms (MPN), polycythemia vera (PV), myeloproliferative dysplastic syndrome, essential thrombocythemia (ET), essential thrombocytosis or myelofibrosis (MF).

20. (canceled)

21. (canceled)

22. (canceled)

23. The method as claimed in claim 18, wherein the disease or condition is selected from small cell lung cancer (SCLC), gastric cancer, pancreatic cancer, lung cancer, liver metastasis and breast cancer.

24. The method as claimed in claim 18, wherein the cancer has RB1 mutation.

25. A method of treating a subject with a tumor, the method comprising: administering to the subject, a therapeutically effective amount of the compound as claimed in claim 1.

26. The method as claimed in claim 25, wherein the tumor is a neuroendocrine tumor, and the neuroendocrine tumor is a small cell lung cancer, neuroendocrine prostate cancer or MYC amplified tumor.

27. (canceled)

28. A method of treating a subject with cancer, the method comprising: administering to the subject a therapeutically effective amount of the compound as claimed in claim 1.
